(19) United States
(12) Patent Application Publication
(10) Pub. No.: US 2026/0131047 A1
(43) Pub. Date: May 14, 2026

(54) TREATMENT SYSTEMS FOR WOUND CARE

(71) Applicant: Peterson Science, Casper, WY (US)

(72) Inventor: William R. Peterson, III, Huntington Beach, CA (US)

(73) Assignee: Peterson Science, Casper, WY (US)

(21) Appl. No.: 19/383,011

(22) Filed: Nov. 7, 2025

Related U.S. Application Data
(60) Provisional application No. 63/718,356, filed on Nov. 8, 2024.

(51) Int. Cl. A61L 26/00

(52) U.S. Cl. CPC A61L 26/0066

(57) ABSTRACT

Various embodiments provide methods for treating a wound. Such methods can comprise the steps of providing an aqueous treatment solution comprising hyaluronic acid, at least one silane, and an organic acid; spraying the aqueous solution onto a wound on the skin surface; crosslinking the hyaluronic acid and the silane to create a biocompatible matrix; and bonding a thin layer of the biocompatible matrix over the wound on the skin surface.

### BACKGROUND OF THE INVENTION

Although current wound treatments can be effective in managing pain and healing the skin, the effectiveness of the treatment can vary depending on the severity of the wound, the type of treatment used, and individual factors such as skin type and overall health. What is needed are new compositions and methods for the treatment of wounds in the skin.

### SUMMARY OF THE INVENTION

Various embodiments, described herein, teach topical applications of an aqueous solution of organosilicon quaternary ammonium compounds and hyaluronic acid to the skin. Some embodiments teach topical application methods utilizing these compositions as a broad-spectrum antimicrobial, which bond to the skin and treat the skin. Some embodiments teach topical application methods utilizing these compositions and a medicant that is configured to treat a skin condition. For example, various embodiments, described herein, provide new and novel compositions and methods for the treatment of wounds.

Various embodiments provide a wound dressing device, comprising a biocompatible matrix comprising hyaluronic acid and at least one silane, wherein the at least one silane is cross-linked to the hyaluronic acid; wherein the biocompatible matrix is insoluble; and wherein the biocompatible matrix is an antimicrobial.

In some embodiments, the wound dressing device can further comprise a woven material. In some examples, the biocompatible matrix is coated onto the woven material. In some examples, the woven material is a gauze. In some examples, the woven material further comprises a bandage.

In some embodiments, the wound dressing device can further comprise an active agent directly incorporated into the biocompatible matrix. In some examples, the active agent is an alcohol. In some examples, the active agent is a medicant. In some embodiments, the biocompatible matrix is a film or a sheet.

Some embodiments provide a composition for wound care, the composition comprising: an organosilane quaternary ammonium compound in in a range from 0.1% to 1% of a weight of the composition; low molecular weight hyaluronic acid in a range from 0.1% to 10% of the weight of the composition; an organic acid in a range from 0.01% to 2% of the weight of the composition; an alcohol in a range from 0.1% to 5% of the weight of the composition; and water in a range from 90% to 99% of the weight of the composition.

Some embodiments provide methods for treating a wound. For example, a method for treating a wound can include: providing a liquid composition comprising hyaluronic acid, at least one silane, salicylic acid, an organic acid, and water; administering the liquid composition onto a skin surface comprising the wound; crosslinking the hyaluronic acid and the at least one silane to create a biocompatible matrix; creating a thin layer of the matrix over the skin surface; and exudating liquid from the skin surface produced by the wound into the biocompatible matrix.

The biocompatible matrix is an antimicrobial. The silane comprises a quaternary ammonium group. In some embodiments, the silane is 1-Octadecanaminium, N,N-dimethyl-N-[3-(trihydroxysilyl) propyl]-chloride.

The spray device can be configured to propel the liquid composition from the container onto the surface of skin. The silane crosslinks with the hyaluronic acid when the liquid composition is on the surface of skin. In some configurations of the system, the at least one silane is octadecyldimethyl (3-triethoxysilylpropyl) ammonium chloride in a weight percentage range from 0.1% to 1%; and wherein the hyaluronic acid has a molecular weight in the range from 5 kDa to 20 kDa, and is in a weight percentage range from 0.01% to 2%.

These embodiments, as well as, other embodiments, additional features, certain configurations, and exemplary implementations, are described in the following drawings and description.

### BRIEF DESCRIPTION OF THE DRAWINGS

The present disclosure will become more fully understood from the description and the accompanying drawings, wherein:

FIG. 1 is an illustration showing an exemplary embodiment of a skin treatment system comprising an exemplary spray device containing a liquid treatment composition.

FIG. 2 is a chemical drawing representing an exemplary hydraulic acid-organosilane crosslinked polymer, according to various embodiments;

FIG. 3 is a cross-sectional side view of an exemplary biocompatible matrix interfaced to a skin surface, according to various embodiments;

FIG. 4 is a cross-sectional side view of an exemplary biocompatible treatment sheet attached to a skin surface over an treatment zone comprising a wound, according to various embodiments;

FIG. 5 is a cross-sectional side view of an exemplary wound treatment system interfaced to a wound into and through a skin surface, according to various embodiments;

FIG. 6 is a cross-sectional side view of an exemplary biocompatible matrix comprising an optical filter, which is interfaced to a skin surface, according to various embodiments;

FIG. 7 is a flow chart describing an exemplary product by process, according to various embodiments;

FIG. 8 is a photograph of exemplary biocompatible treatment sheet, according to various embodiments;

FIG. 9 is a photograph of exemplary biocompatible treatment sheet, according to various embodiments;

FIGS. 10A-10D are a series of photographs illustrating results of treatment of a wound on a hand, according to various embodiments;

FIG. 11A-11K are a series of photographs illustrating results of treatment of a wound on a hand, according to various embodiments;

FIGS. 12A-12H are a series of photographs illustrating results of treatment of a wound on a dog, according to various embodiments; and

FIG. 13A-13N are a series of photographs illustrating results of treatment of a wound on a back, according to various embodiments.

The drawings described herein are for illustrative purposes only of selected embodiments and are not intended to represent all possible implementations and are not intended to limit the scope of any of the exemplary embodiments disclosed herein or any equivalents thereof.

### DETAILED DESCRIPTION OF THE INVENTION

The following description is merely exemplary in nature and is in no way intended to limit the exemplary embodiments, their applications, or uses. It is understood that the steps within a method may be executed in different order without altering the principles of the present disclosure. For example, various embodiments may be described herein in terms of various functional components and processing steps. It should be appreciated that such components and steps may be realized by any number of hardware components configured to perform the specified functions.

Various embodiments provide methods for treating a skin surface. Such methods can comprise the steps of providing an aqueous treatment solution comprising hyaluronic acid, at least one silane, and an organic acid; spraying the aqueous solution onto a treatment area on the skin surface; crosslinking the hyaluronic acid and the silane to create a biocompatible matrix; and bonding a thin layer of the biocompatible matrix over the treatment area of the skin surface.

Various embodiments provide methods for treating a wound. A method of treating wound can comprise the steps of providing a liquid composition comprising hyaluronic acid, at least one silane, an organic acid, and water; applying the liquid composition onto a skin surface comprising the wound; crosslinking the hyaluronic acid and the at least one silane to create a biocompatible matrix; bonding the biocompatible matrix over the skin surface comprising the wound; moisturizing the skin surface; preventing trans epidermal water loss from the skin surface; and administering an anti-microbial to the wound. The methods can include delivering a medicant to the wound.

The biocompatible matrix can be a antimicrobial. The silane can comprise a quaternary ammonium group. The liquid composition can include salicylic acid. The liquid composition can include alcohol.

The liquid composition can comprise silane in a range from 0.1% to 1% of a weight of the composition, hyaluronic acid in a range from 0.01% to 2% of the weight of the composition, organic acid in a range from 0.001% to 2% of the weight of the composition, and water in a range from 70% to 99% of the weight of the composition.

Various embodiments provide a novel aqueous wound care technique that auto-assembles an antimicrobial hyaluronan hydrogel that covalently bonds with wet or dry tissue. The biocompatible matrix promotes wound healing, reduces inflammation, deliverers constant moisturization to the wound site reducing transepidermal water loss (TEWL), and mitigates infection utilizing a persistent non leaching and non-migratory antimicrobial system that is bound throughout the matrix.

The resultant biocompatible matrix provides a breathable platform that allows for gas transmission from the skin surface. This breathable platform allows a compound (for example, a medicant) mixed into the hydrogel to be transferred from the hydrogel to the skin surface. This application may be applied continuously through the wound healing process.

Various embodiments provide a skin treatment system. An exemplary skin treatment system can include a liquid composition comprising hyaluronic acid, at least one silane, salicylic acid, an organic acid, and water; a container configured to hold the liquid composition; and a delivery device coupled to the container and in communication with the liquid.

The delivery device is configured to apply the liquid composition from the container onto a surface of skin. The delivery device can be one of a facial pad, a wipe, a towelette, a roller ball, a mister, an aerosol dispenser, and an atomizer. Upon application by the delivery device, the silane crosslinks with the hyaluronic acid and the skin when the liquid composition bonds to the surface of skin.

The liquid composition can comprise hyaluronic acid has a molecular weight in the range from 5 kDa to 300 kDa; and silane is octadecyldimethyl (3-triethoxysilylpropyl) ammonium chloride.

The liquid composition can comprise a weight percentage of the hyaluronic acid is in a range from 0.01% to 2%; a weight percentage of the silane is in a range from 0.1% to 1%; a weight percentage of the salicylic acid is in a range from 0.1% to 3%; a weight percentage of the organic acid is acid is in a range from 0.001% to 2%; and a weight percentage of the water is in a range from 70% to 98%.

The organic acid is one of acetic acid, carboxylic acid, citric acid, formic acid, lactic acid, malic acid, oxalic acid, tartaric acid, uric acid, and combinations thereof. The organic acid has a pKa in a range from 2.5 to 6.5.

The liquid composition can further comprise an alcohol in a range from 0.1% to 15% the weight of the liquid composition. The alcohol can be one of methanol, ethanol, propanol, isopropyl, and combinations thereof.

The liquid composition can further comprise a medicant. The medicant can be one of benzoyl peroxide, salicylic acid, topical retinoids, antibiotics, hormones, isotretinoin, and combinations thereof.

### Treatment System

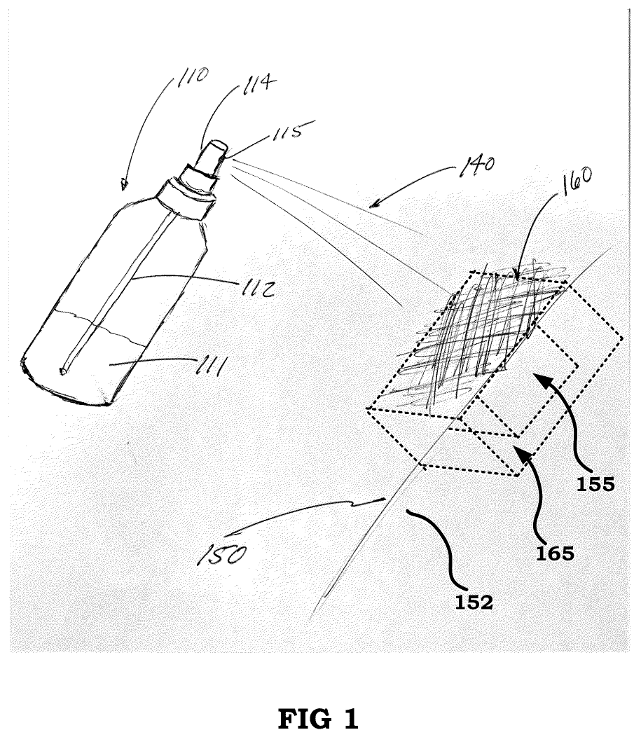

Turning to FIG. 1, a skin treatment system 100 can comprise an exemplary spray device 110 containing a liquid treatment composition 111. The exemplary spray device 110 can comprise a bottle 105, which holds the liquid treatment composition 111, a pump cap 114, and a tube 112 in fluid communication with the liquid treatment composition 111 and a nozzle 115 in the pump cap 144. When the pump cap 114 of the skin treatment system 100 is activated, an aerosol 140 of a portion of the liquid treatment composition 111 to a skin surface 150. Upon landing on the skin surface 150, the liquid treatment composition 111 crosslinks to create a biocompatible matrix 160, which can bond to the skin surface 150. Various components in the biocompatible matrix 160 treat a skin condition in an infection zone 155 on the skin surface 150, as further discussed below.

A method of treating a skin condition can include the steps of spraying the liquid treatment composition 111 onto a treatment area on the skin surface 150, crosslinking the liquid treatment composition 111 to create a biocompatible matrix 160, and bonding a thin layer of the biocompatible matrix 160 over a treatment area 165 of the skin surface 150.

For effective treatment of the skin condition, the treatment area 165 encompasses the infection zone 155. The treatment area 165 can be a two-dimensional target for aiming the aerosol 140 of a portion of the liquid treatment composition 111 to a skin surface 150.

Although the treatment area 165 can be three-dimensional and include a portion of the subdermal 152, as illustrated by the hashed lines. For most infection zones 165, the treatment area 165 includes both the skin surface 150 and the subdermal 152. For an infection zone 155, which is limited to the skin surface 150, the treatment area 165 can be limited to the skin surface 150, however, if the treatment area is limited to the skin surface 150, the biocompatible matrix 160 can be configured to provide one or more of a medicant, an emollient, an astringent, a humectant, an antibiotic, and an anti-inflammatory, to a subdermal 152 region below the treatment area 165. The biocompatible matrix 160 is an antimicrobial.

Exemplary skin conditions that can be treated with the skin treatment system 100 can include, but are not limited to, wounds, fungal infections, such as athlete's foot and jock itch, bacterial infections, seborrheic dermatitis, and skin dryness. In addition, the skin treatment system 100 can contain different formulations the liquid treatment composition 111, which are not directed to skin treatments. For example, the liquid treatment composition 111 can be formulated for use as a hand sanitizer, a skin moisturizer, a sun block barrier, or combinations thereof.

The liquid treatment composition 111 comprises hyaluronic acid, a silane, an organic acid, and water. The liquid treatment composition 111 is an aqueous solution. The liquid treatment composition 111 can further comprise various medicants, emulsifiers, fragrances, emollients, astringents, humectants, and alcohols.

Hyaluronic acid (“HA”), also known as hyaluronan, is a substance naturally found in the human body. Polymers of HA can range in size from 4 kDa to 60,000 kDa, Typically, HA is categorized into high molecular weight (“HMW”) HA and low molecular weight (“LMW”) HA. The HMW-HA is a very large molecule and the natural form of HA.

The LMW-HA has a molecular weight in the range from 5 kDa to 100 kDa. As known to those of skill in the art, hyaluronan is a polymer, and the subunits can be repeated many times. However, the LMW-HA is hydrolyzed in an aqueous solution with the subunits being repeated only a few times. To further enable the inventions described herein, sources of LMW-HA that have been used in preparing exemplary mixtures of the liquid treatment composition 111 are listed below.

Without being bound by theory, the smaller LMW-HA fragments remain stable in the liquid treatment solution 111 until the solution is applied to a substrate, such as a skin surface 150. The stability in the treatment solution 111 in the treatment system 110 is a result of the smaller hydrolyzed LMW-HA fragments having fewer hydroxylated subunits to crosslink with and the amount of water in the solution 111 which prevents polymerization of silanol functional group on the organosiloxane by a condensation reaction.

The LMW-HA in aqueous solutions can self-associate to form transient clusters in solution. While LMW-HA is considered a polyelectrolyte polymer chain, LMW-HA does not exhibit the polyelectrolyte peak, suggesting the absence of a characteristic length scale between the LMW-HA molecules and the emergence of a fractal clustering, which is due to the strong solvation of these molecules.

However, the silanol moieties are very reactive and will react through a condensation reaction with a wide variety of hydroxylated surfaces. Upon application of the treatment solution 111 to a substrate, for example the skin surface 150, the silanol functional groups undergo condensation reactions with other silanol functional groups, the hydroxides on the LMW-HA, and with the skin surface 150, thus displacing the remaining aqueous solution, which evaporates into the surrounding air. The condensation reaction occurs across the skin surface 150 to produce a biocompatible matrix 160 having 3-dimensional structure of cross-linked polymers. The biocompatible matrix 160 is bound to the skin surface 150 and is typically between 10 and 20 molecules thick.

In some embodiments, the LMW-HA forms a hydrogel and the biocompatible matrix 160 comprises the hydrogel crosslinked to a plurality of organosiloxane molecules. In some aspects of such embodiments, the hydrogel can further comprise one or more of a medicant, an emollient, an astringent, a humectant, an antibiotic, and an anti-inflammatory.

In some embodiments, the organosiloxine is an organosilane quaternary ammonium. In certain embodiments, the organosiloxine contains an ammonium halide and a hydrolyzable alkoxy group bonded to silicon. Examples of the organosilane quaternary ammonium compounds for the biocompatible matrix 160 include, but are not limited to, 3-(trimethoxysilyl) propyloctadecyldimethyl ammonium chloride, 3-(trimethoxysilyl)-propyldidecylmethyl ammonium chloride, the trisilanol derivatives and the polysiloxanol derivatives and mixtures thereof. Other examples of quaternary ammonium organosilicon compounds, the silanol derivatives and mixtures thereof, such as 3-(trimethoxysilyl)-propyldimethyltetradecyl ammonium chloride, 3-(trimethoxysilyl) propyldimethylhexadecyl ammonium chloride, 3-(dimethoxymethylsilyl) propyldimethyloctadecyl ammonium chloride and 3-(methoxydimethylsilyl) propyldimethyloctadecyl ammonium chloride may also be employed for the biocompatible matrix 160, as would be apparent to those skilled in the art. In certain embodiments, the organosiloxine is octadecyldimethyl (3-triethoxysilylpropyl) ammonium chloride.

Spraying the treatment solution 111 on to the skin surface 150 is a desirable method of application of the treatment solution 111. By spraying the treatment solution 111, a thin layer of the solution 111 covers a treatment area of skin surface 150 and the aerosol produced by the spray removes enough water from the skin surface 150 to allow the condensation reaction to commence and cross link the biocompatible matrix 160 to the treatment area of the skin surface 150.

In some embodiments, a silane compound crosslinks with the LMW-HA upon the treatment composition 111 being applied to a surface of skin 150. In certain embodiments, the organosiloxine compound is an organosilane quaternary ammonium compound.

For example, when applied to skin, the treatment composition 111 containing the trihydrosilane and LMW-HA forms a layer on the surface on the skin 150, in which the hydroxy groups attached to the silicon moiety orient toward the skin 150 and form weak, electrostatic hydrogen bonds. As the aqueous volatilizes, the reactive silanol moieties begin reacting with their adjacent molecules and with the LMW-HA, splitting out water molecules to form siloxane bonds. Additionally, the silanols form covalent bonds at this time with the LMW-HA crosslinking the material into a hydrogel and to the skin while also splitting out water. The covalent bonds formed within the crosslinked LMW-HA hydrogel and the skin 150 are extremely strong and cannot be removed by water or solvents. Without being bound by theory, it is the covalent bonding present between the quat moieties, the LMW HA, and the bonding to the skin surface that promotes the stability and insolubility of the resulting biocompatible matrix 160. The biocompatible matrix 160 is now antimicrobially active due to the high number of active quaternary ammonium structures within the crosslinked LMW-HA hydrogel and on the surface of the skin 150.

Accordingly, the biocompatible matrix 160 is an antimicrobial. For example, quaternary ammonium organosilanes have antimicrobial properties of against a wide range of pathogens including, but not limited to: Citrobacter freundii, Citrobacter diversus, Corynebacterium diptheriae, Diplococcus pneumoniae, Micrococcus sp. (I), Micrococcus sp. (II), Micrococcus sp. (III), Mycobacterium spp., Staphylococcus albus, Staphylococcus aureus, Staphylococcus citrens, Staphylococcus epidermidis, Streptococcus faecalis, Streptococcus pyogenes, Acinetobacter calcoaceticus, Enterobacter aerogenes, Enterobacter aglomerans (I), Enterobacter aglomerans (II), Escherichia coli, Klebsiella pneumoniae, Nisseria gonorrhoeae, Proteus mirabilis, Proteus morganii, Proteus vulgaris, Providencia spp., Pseudomonas, Pseudomonas aeruginosa, Pseudomonas fragi, Salmonella choleraesuis, Salmonella enteritidis, Salmonella gallinarum, Salmonella paratyphi A, Salmonella schottmuelleri, Salmonella typhimurium, Salmonella typhosa, Serratia marcescens, Shigella flexnerie Type II, Shigella sonnei, Virbrio cholerae, Adenovirus Type IV, Feline Pneumonitis, Herpes Simplex Type I & II, HIV-1 (AIDS), Influenza A (Japan), Influenza A2 (Aichi), Influenza A2 (Hong Kong), Parinfluenza (Sendai), Poliovirus, Reovirus, Respiratory Synctia, Alternaria alternata, Aspergillus niger, Aureobasidium pullulans, Candida albicans, Cladosporium cladosporioides, Drechslera australiensis, Gliomastix cerealis, Microsporum audouinii, Monilia grisea, Phoma fimeti, Pithomyces chartarum, Scolecobasidium humicola, Trychophyton interdigitale, and Trychophyton mentagrophytes.

In addition, the EPA has registered the active ingredient Octadecyldimethyltrihydroxysilyl propyl ammonium chloride as an antimicrobial against bacteria, fungi, mold, mildew, and algae. Tests on animal subjects have shown that Octadecyldimethyltrihydroxysilyl propyl ammonium chloride does not irritate the skin (guinea pigs and rabbits) or eyes (rabbits). Moreover, tests on animal subjects have shown that Octadecyldimethyltrihydroxysilyl propyl ammonium chloride is not toxic via ingestion (LD₅₀>5000 mg/kg), skin (LD₅₀>5050 mg/kg), or inhalation (LD₅₀>2 mg/L). The EPA has registered the active ingredient Octadecyldimethyltrihydroxysilyl propyl ammonium chloride as an antiviral and has included this active ingredient in EPA List N: Disinfectants for Use Against SARS-CoV-2.

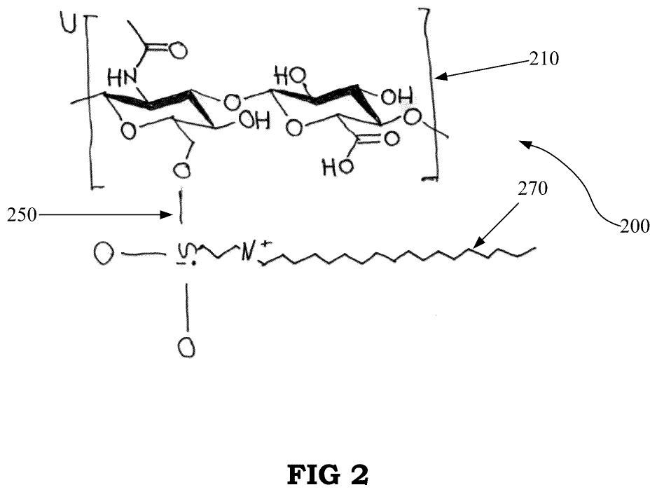

As illustrated in FIG. 2, the biocompatible matrix 200 comprises LMW-HA polymeric unit 210 crosslinked 250 to organosilane quaternary ammonium compound 270. The biocompatible matrix 200 can comprise a polymer of thousands of LMW-HA polymeric units 210, each crosslinked 250 to one or more molecules of the organosilane quaternary ammonium compound 270. In some configurations the polymer of thousands of LMW-HA polymeric units 210 can be a hydrogel.

The cross-linking process involves creating covalent bonds between different organosiloxane molecules to form a three-dimensional network or polymer. For example, organosiloxanes containing reactive groups, such as hydroxyl (OH) or alkoxy (OR) groups, can undergo condensation reactions to form cross-links. This is typically achieved by adding a cross-linking agent, such as a silane or siloxane with multiple reactive groups, to the organosiloxane mixture. The reactive groups on the cross-linking agent react with the reactive groups on the organosiloxane, forming new siloxane bonds and resulting in cross-linking.

In condensation reactions, the condensation product will become a linear polymer if both moieties are difunctional. If the moieties are tri- or tetra functional, the result will be a crosslinked polymer, which is a three-dimensional network. In some condensation reactions, the organosiloxane molecules are in an aqueous solution and water is displaced by the polymerization process. The displaced water can evaporate into the environment yielding the condensation product on a substrate. As described herein, various organosiloxanes are tri- or tetra functional. LMW-HA can be tri- or tetra functional, which can be modified by, for example, a solution that comprises the LMW-HA, or by a material that dopes the LMW-HA.

In some embodiments, when applied to skin, the aqueous solution containing the trihydrosilane forms a layer on the surface in which the hydroxy groups attached to the silicon moiety orient toward the skin and form weak, electrostatic hydrogen bonds. As the aqueous volatilizes, the reactive silanol moieties begin reacting with their adjacent molecules, splitting out water molecules to form siloxane bonds. This process, 2 Si—OH forms Si—O—Si and H2O. Additionally, the silanols form covalent bonds at this time to the substrate/skin while also splitting out water. The covalent bonds formed are extremely strong and cannot be removed by water or solvents. The strength of a Si—O bond is approximately 128 kcal/mole (hence the stability of quartz). It is the covalent bonding present between the quat moieties and the bonding to the substrate surface that promotes the stability and insolubility of the resulting film. As it is bonded to the surface it becomes non-leaching when exposed to water or solvents. Even when removed from a substrate surface (abrasion, etc.) it exists in a silsesquioxane form that is also extremely insoluble and yet still extremely antimicrobially active due to the high number of active quat structures on the surface. As opposed to the water solution of the active, the density of the active sites of the silsesquioxane film per unit area are millions of times higher.

In another example, organosiloxanes with unsaturated organic groups, such as vinyl or allyl groups, can undergo addition reactions to form cross-links. This process often involves the use of a catalyst, such as a platinum-based catalyst, which promotes the addition of the unsaturated groups across the silicon-oxygen bonds. The addition reactions lead to the formation of new siloxane bonds, resulting in cross-linking.

Many other methods of crosslinking organosiloxanes are known to those of skill in the art. Selection of an appropriate method of crosslinking depends on factors, such as, the specific organosiloxane formulation, desired properties, degree of crosslinking, mechanical strength, and the application requirements.

In some embodiments, the silanol moieties of the organosilane compound 270 are very reactive and will react through a condensation reaction with a wide variety of hydroxylated surfaces of the LMW-HA polymeric units 210. The silanols undergo autocondensation reactions with other silanol substituted molecules, the hydroxides on the LMW-HA, and with the surface of the skin eliminating water. Autocondensation occurs across the surface of skin to produce 3-dimensional, cross-linked polymers being on the average of 10-20 molecules thick.

Those of skill in the art understand crosslinking the LMW-HA polymeric units 210 to yield desirable properties, which is discussed in, for example, Sanchez-Tellez, D A, et. al., “Siloxane-inorganic chemical crosslinking of hyaluronic acid—based hybrid hydrogels: Structural characterization.” Carbohydrate Polymers 230 (2020) 115590.

Molecules that have not bonded with the silanols are free to pass through the skin. Examples of such molecules include but are not limited to a medicant, an emollient, an astringent, a humectant, an antibiotic, and an anti-inflammatory.

### Biocompatible Matrix

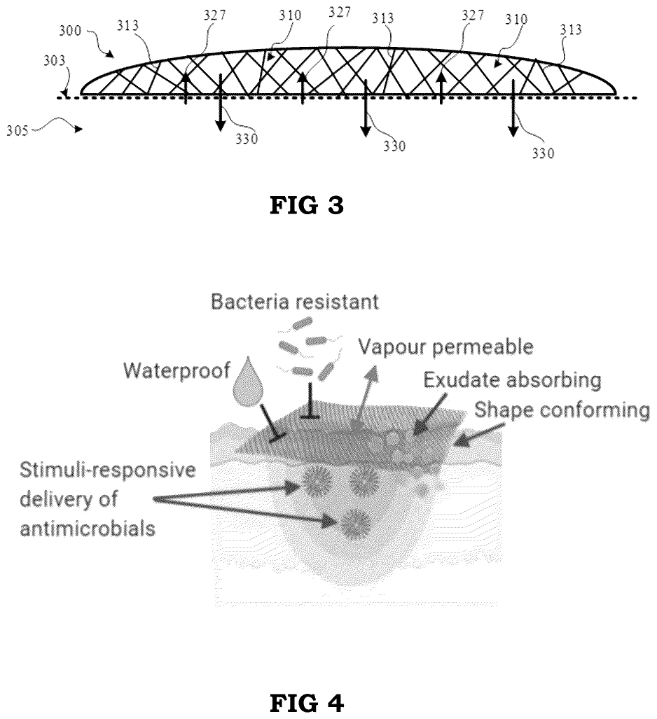

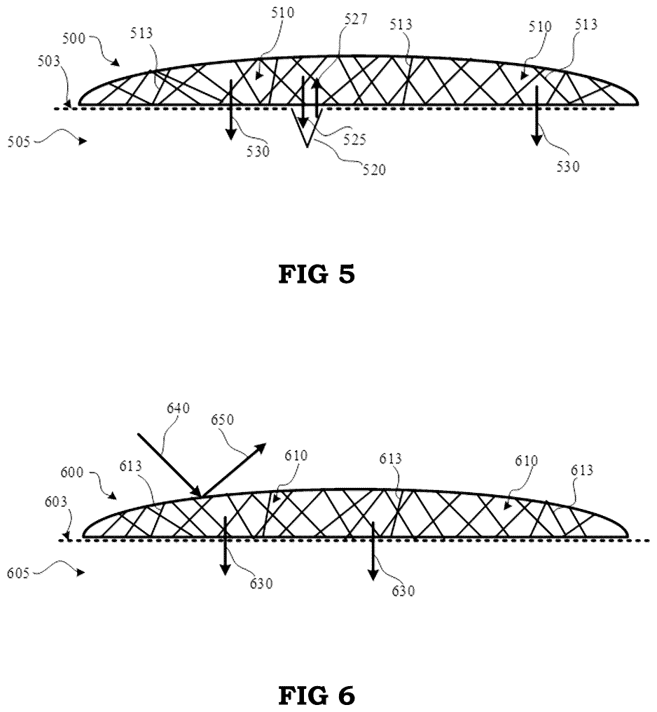

In FIGS. 3-6, examples of the treatment of various skin conditions using a biocompatible matrix are illustrated. As will be obvious to those of skill in the art, the various features, additional components, and medicants described in any of the examples illustrated in FIGS. 3-6 and described herein, can be used in any other example that has been described in a different FIG. 3-6. For example, the one or more sun blocking agents in the sunscreen matrix 600 of FIG. 6, can be incorporated into any of the biocompatible matrix described in FIGS. 3-5. In another example, the wound healing agents in the LWM-HA of the wound dressing 500 of FIG. 5 can be incorporated into any of the biocompatible matrix described in FIGS. 3-4 and 6.

With reference to FIG. 3, an exemplary biocompatible matrix 300 is attached to a skin surface 303. This exemplary biocompatible matrix 300 comprises at least a hydrogel 310 crosslinked to a plurality of siloxane molecules 313. The hydrogel 310 comprises LMW HA. The plurality of siloxane molecules 313 can form a polymer, which is crosslinked to the hydrogel 310. Some of the siloxane molecules 313 can bind to the skin surface 303, as discussed herein.

In some configurations of the biocompatible matrix 300, the hydrogel 310 can comprise one or more additional components. In example configurations, the additional components can include, but are not limited to, medicants, emollients, astringents, humectants, scents, flavorings, preservatives, surfactants, bone scaffolding, metal ions, acids, alcohols, antibiotics, vitamins, hormones, antioxidants, and anti-inflammatories.

One or more of the additional components can include one or more active ingredients. Examples of active ingredients in the biocompatible matrix 300 can include a first additional component 330 and a second additional component 327 As illustrated in FIG. 3, the first additional component 330 can move from the hydrogel 310 onto the skin surface 303 and then can absorbed into the subdermal 305. As further illustrated in FIG. 3, the second additional component 327 can absorb a blemish from an infection zone into the hydrogel 310. As used herein, a blemish can include, but not limited to, oil, dry skin, exudate, blood, crud, dirt, bacteria, fungus, or any other material produced by a skin ailment or wound.

In some embodiments, the first additional component 330 can include a medicant. In addition, the LMW-HA in the hydrogel 310 can be a moisturizer, as further described in FIG. 6.

For example, seborrheic dermatitis is a chronic inflammatory skin disorder that affects sebum rich areas of the body. It is one of the most common skin diseases, with a prevalence in middle age and elderly populations. seborrheic dermatitis follows a relapse and remitting course, worsening with stress and the winter months. The underlying mechanism of seborrheic dermatitis is poorly understood. It is believed that a combination of Malassezia (skin floral yeast found on 90% of adults) species colonization, immune system activation, and a genetic predisposition. Among various endogenous and exogenous factors that likely contribute to it pathogenesis.

However, several medicants for seborrheic dermatitis have been proven to be effective in managing and reducing seborrheic dermatitis symptoms. For example, the medicant can be topical antifungals, topical calcineurin inhibitors, corticosteroids, and combinations thereof.

Topical antifungals such as ketoconazole and ciclopirox (Loprox) work by destroying the fungus associated with seborrheic dermatitis reducing scaling and inflammation. Calcineurin inhibitors can help to suppress inflammatory overactivity by acting on the T-cells to dampen down the immune system. This helps to reduce inflammation and make the skin less itchy. Corticosteroids work by decreasing inflammation and the activity of the immune system.

With reference to FIG. 4, an treatment sheet is attached to a skin surface over an treatment zone comprising a wound. In various embodiments, the biocompatible treatment sheet is waterproof, as well as vapor permeable. The treatment sheet can be exudate absorbing. The treatment sheet is bacteria resistant and in some embodiments is an anti-microbial. In some configurations, the treatment sheet can deliver antimicrobials and/or medicants into the skin. The treatment sheet is shape conforming to the surface of the skin. In some embodiments, the treatment sheet has an adhesive on one surface for adhering the treatment sheet to the skin. In some embodiments, the treatment sheet adheres to the skin by static electricity or by chemical interaction with the surface of the skin.

Inflammation is a natural response to injury, but it can also prolong the healing process if it becomes excessive. Hyaluronic acid has anti-inflammatory properties that can help to reduce inflammation at the wound site, leading to a faster healing time. Hyaluronic acid is known for its ability to attract and hold onto water molecules, which can help to keep the wound bed moist. This moist environment promotes healing by creating an optimal environment for cell growth and tissue repair.

Hyaluronic acid helps to create a matrix or scaffolding that supports the growth of new cells and tissues. Hyaluronic acid promotes cell migration and proliferation, which helps to speed up the healing process.

Hyaluronic acid can form a protective barrier over the wound, which can help to prevent infection and further damage. This barrier also helps to keep the wound bed moist and supports the healing process. Hyaluronic acid is not typically used as a primary treatment for wounds. It is primarily known for its role in skincare and joint health. In wound healing, other treatments and approaches are more commonly employed.

In some configurations of the treatment sheet the hydrogel can comprise one or more additional components. In example configurations, the additional components can include, but are not limited to, medicants, emollients, astringents, humectants, scents, flavorings, preservatives, surfactants, bone scaffolding, metal ions, acids, alcohols, antibiotics, vitamins, hormones, antioxidants, and anti-inflammatories.

In some embodiments, the treatment sheet can comprise a medicant, such as, but not limited to various medicants described herein. For example, the medicant can be benzoyl peroxide, salicylic acid, topical retinoids, antibiotics, hormones, isotretinoin, and combinations thereof.

Without being bound by theory, treatment matrix sheet can treat a wound in multiple ways. The siloxane molecules create a persistent non-leaching and non-migratory antimicrobial with occlusive moisturizing properties to help the skin retain moisture and continuously destroy the bacteria associated with the wound.

Without being bound by theory, the HMW-HA hydrogel in the treatment sheet can function as a long-lasting humectant moisturizer that helps skin, in the treatment area, retain moisture and heal inflammation without clogging overactive pores.

The hydrogel in the treatment sheet provides a breathable platform, which allows gas transmission from the skin surface to exit through the sheet. This breathable platform allows a compound (for example, a medicant) mixed into the hydrogel to be transferred from the hydrogel to the skin surface.

The HWM-HA in the hydrogel can provide wound healing agents to the wound and the subdermal surrounding the wound. Exemplary wound healing agents can include growth factors, mucopolysaccharides and proteins. The wound healing agents provided by the HMW-HA are further discussed herein. In some methods of treating a wound, the hydrogel provides both a medicant and at least one wound healing agent to treatment area that includes the wound.

The hydrogel can absorb an exudate excreted by the wound. The hydrogel is a strong adsorbent capable of handling the high level of exudates from the wound. The treatment sheet insulates the exudate without adhering to the wound and provides a moisturizer to the skin surface. Those of skill in the art can tune the hydrogel to improve an absorption of the exudate, which is discussed in, for example, Firlar I, et al., “Functional Hydrogels for Treatment of Chronic Wounds”. Gels. 2022 Feb. 17; 8 (2): 127.

The hydrogel can be tuned to absorb fluids and gel-like molecules and balance environment moisture on the skin surface. The hydrogel can provide autolytic debridement, soften and loosen necrosis, rehydrate wound bed, provide moisture onto wound and surrounding skin, be tuned to absorb exudate and lock away it from the wound, including the exudate along with its harmful components, salts, toxins, bacteria, and liquefied debris. This prevents the exudate from transferring to other areas of the skin surface and protects vulnerable skin surface around the wound.

With reference to FIG. 5, a wound dressing 500 is attached to a skin surface 503 over a wound 520 through the skin surface 503. The wound 520 can penetrate into the subdermal 505. The wound 520 can be an abrasion, a cut, a gash, a bullet hole, or the like. However, in some embodiments, the wound 520 is a rash, an infection, a burn, or other skin surface ailment.

This wound dressing 500 comprises at least a hydrogel 510 crosslinked to a plurality of siloxane molecules 513. The hydrogel 510 comprises LMW-HA. The plurality of siloxane molecules 513 can form a polymer, which is crosslinked to the hydrogel 510. Some of the siloxane molecules 513 can bind to the skin surface 503. The wound dressing 500 is an antimicrobial.

In some configurations of the wound dressing 500, the hydrogel 510 can comprise one or more additional components. In example configurations, the additional components can include, but are not limited to, medicants, emollients, astringents, humectants, scents, flavorings, preservatives, surfactants, bone scaffolding, metal ions, acids, alcohols, antibiotics, vitamins, hormones, antioxidants, and anti-inflammatories.

The treatment composition can comprise an alcohol as a medicant for wound care sprays. The alcohol decreases the drying time of the wound dressing 500 and allows for more even monolayer formation on the skin surface 503. In addition the alcohol provides the treatment area “instant” antimicrobial properties, which disinfects the wound 520.

In some methods, the step of treating a wound 520 in the skin surface 503, include sealing the wound 520, disinfecting the wound 520, and promote healing of the wound 520.

For example, sealing the wound 520 can include spraying an aqueous treatment composition over the wound 520 and onto the skin surface 503 surrounding the wound 520. Crosslinking the aqueous treatment composition to create the wound dressing 500 and bonding a thin layer of the wound dressing 500 onto the skin surface 503 surrounding the wound 520. The wound dressing 500 seals the wound 520, which can reduce or stop bleeding from the wound 520. In addition, the wound dressing 500 can be applied continuously during the wound 520 healing process, which can seal the wound 520 from dirt, germs, infection, and the like, which can irritate the wound 520 and/or prolong the healing process of the wound 520.

The disinfecting the wound 520 can include applying the antimicrobial properties of the wound dressing 500, as described by the treatment compositions herein. In some configurations, the wound dressing 500 contains a medicant and administers the medicant into the wound 520 the skin surface 503 surrounding the wound 520. The medicant 525 can be any individual or combination of the antimicrobial agents, antifungal agents, and antiviral agents, which have been described herein. In this configuration, the medicant 525 disinfects the wound 520 the skin surface 503 surrounding the wound 520 each time a wound dressing 500 is applied to the wound 520.

The LMW-HA in the wound dressing can promote healing of the wound 520. As a major component of the extracellular matrix, hyaluronic acid has a key role in tissue regeneration, inflammation response, and angiogenesis, which are phases of wound repair. It is well known that HA exhibits several beneficial effects on wound healing, such as the decrease of inflammatory processes, regulation of tissue remodeling, and enhancement of angiogenesis. The design of this novel wound dressing 500, encompassing hydrogels has beneficial effects on wound healing. Those of skill in the art can tune the hydrogel 510 to improve the healing properties of the wound dressing 500, which is discussed in, for example, Dovedytis, M., et. al., “Hyaluronic acid and its biomedical applications: A Review.”, Engineered Regeneration 1 (2020) 102-113.

Immediately after a skin injury occurs, the healing process begins to re-establish, as soon as possible, the skin tissue architecture as well as halt the bleeding To accomplish that, platelets release large amounts of HMW-HA, that prompt the deposition of fibrinogen and formation of an initial clot. Further, HA, as a major component of the edema fluid, also promotes the recruitment of neutrophils cells, involved in the phagocytosis of the debris and removal of dead tissue, and the subsequent release of tumor necrosis factor-alpha (TNF-α), IL-1B, IL-8. Further, the secretion of inflammatory cytokines will also contribute to HMW-HA fragmentation into LMW-HA, which is involved in the recruitment of leucocytes and monocytes, a process that is triggered by the binding of HA to the CD44 receptors available on monocytes and granulocytes' surface.

In the last stage of the inflammatory phase, the lymphocytes and macrophages migrate into the wound site, where their toll-like receptors (TLR2 and TLR4) interact with LMW-HA fragments and prompt the expression of TNF-α and interleukins such as IL-6, IL-8 and IL-1β. In addition, LMW-HA together with fibronectin guide the fibroblasts invasion and proliferation, which is mandatory for collagen deposition within the wound, as well as promotes the differentiation of fibroblasts into myofibroblasts (cells ex-pressing smooth-muscle actin and myosin), that play a pivotal role in the wound contraction. Moreover, it has been demonstrated that LMW-HA fragments composed of 6-20 disaccharides can stimulate dermal fibroblast migration and proliferation, with the subsequent deposition of type III collagen, leading to the formation of a new extracellular matrix. Those of skill in the art can find discussion of TLR2 and TLR4 interaction with LMW-HA fragments, in for example, Gariboldi et. al., “Low Molecular Weight Hyaluronic Acid Increases the Self-Defense of Skin Epithelium by Induction of -Defensin 2via TLR2 and TLR4.” Journal of Immunology (2008) 181 (3): 2103-2110.

Moreover, in the re-epithelialization phase, CD44 receptors available in keratinocytes cells interact with the LMW-HA present at the wound margins, regulating the re-epithelialization process.

For example, the LWM-HA in the wound dressing 500 can provide wound healing agents to the treatment area. Exemplary wound healing agents can include growth factors, mucopolysaccharides and proteins. Exemplary growth factor is selected can include one or more of fibroblast growth factor (FGF), acidic fibroblast growth factor (aFGF), nerve growth factor (NGF), epidermal growth factor (EGF), insulin-like growth factors 1 and 2, (IGF-1 and IGF-2), platelet derived growth factor (PDGF), tumor angiogenesis factor (TAF), vascular endothelial growth factor (VEGF), corticotropin releasing factor (CRF), transforming growth factors α and β (TGF-α and TGF-β), interleukin-8 (IL-8); granulocyte-macrophage colony stimulating factor (GM-CSF), interleukins, and interferons.

Those of skill in the art can tune the hydrogel 510 to improve the delivery of healing factors, which is discussed in, for example, Graca M, et. al., “Hyaluronic acid-Based wound dressings: A review.” Carbohydrate Polymers 241 (2020) 116364.

In some embodiments, the wound dressing 500 can comprise a medicant 525. The medicant 525 can be an active agent, which can include, but not limited to, antimicrobial agents, antifungal agents, antiviral agents, and wound healing agents.

A medicant can comprise a metal. A medicant can be a metallic compound. For example, the metallic compound can be a salt of an active agent. The metallic compound can be a salt of an acid. In another example, a metallic ion of the metallic compound is an active ingredient. The metal compound can comprise a metal that changes oxidation state when the metal compound is used in treatment.

In some embodiments, one or more medicants 525 are incorporated directly into micro-cavities of the hydrogel 510 of the wound dressing 500. The medicants 525 may be incorporated by absorption of medicants 525 by the hydrogel 510, and by incorporation during the polymerization of the wound dressing 500. Without being bound by theory, the release of the medicants 525 may be controlled via manipulation of concentration parameters, movement of water through the hydrogel 510 and the degree of cross linking in the wound dressing 500.

The LMW-HA in hydrogel 510 can be tuned for medicant 525 vehiculization into the dermis 505 and controlled release of the medicant 525. The tuning of the LMW-HA for active ingredient transportation is well known in the art and includes parameters of the hydrogel 510, such as, but not limited to, water content, porousness, size of micro-cavities, pH, crosslinking, adding a metal ion, and the like. Those of skill in the art tune the hydrogel 510 to improve the transdermal delivery of the medicant 525, which is discussed in, for example, Zagórska-Dziok, et al. “Hydrogel-Based Active Substance Release Systems for Cosmetology and Dermatology Application: A Review.” Pharmaceutics 2020, 12 (5), 396.

The hydrogel 510 absorbs wound exudate and transfers it into the wound dressing 500 for easy removal from the skin surface 503. In addition, as discussed herein, the hydrogel 510 provides moisture to the wound 520 and surrounding skin surface 503. Bioadhesivity of the wound dressing 500 to the skin surface 503 stabilizes the wound dressing 500 around the wound 520, keeps the wound 520 moist, and absorbs the exudates from the wound 520 during healing. Since the prolonged healing of chronic wounds can increase the risk of infection which adversely affect the healing process, antimicrobial properties of the wound dressing 500 prevents such infections.

In various embodiments, the wound dressing 500 has efficient clinical performance in one of the following: maintaining moisture in the wound environment while absorbing or eliminating excess fluids and exudates, allowing gas transmission, protecting against microbial invasion, providing a barrier to protect the wound from external trauma, and keeping cells viable and decreasing surface necrosis.

In some aspects of treating a wound 520, the treatment composition can be sprayed over the wound 520 and onto the skin surface 503 surrounding the wound 520 of a user (patient) to form a coating (wound dressing 500) that covers the wound 520 and is attached to the skin surface 503 surrounding the wound 520, which is similar to the treatment illustrated in FIG. 1 and as described herein. The organosilane quaternary ammonium compound crosslinks with the LMW-HA upon the treatment composition being applied to the skin surface 503.

With reference to FIG. 6, a cross-sectional side view illustrates an exemplary biocompatible matrix comprising an optical filter, which is interfaced to a skin surface, according to various embodiments. This exemplary sunscreen matrix 600 comprises at least a hydrogel 610 crosslinked to a plurality of siloxane molecules 613. The hydrogel 610 comprises LMW HA. The plurality of siloxane molecules 613 can form a polymer, which is crosslinked to the hydrogel 610. Some of the siloxane molecules 613 can bind to the skin surface 603, as discussed herein.

In some configurations of the sunscreen matrix 600, the hydrogel 610 can comprise one or more sun blocking agents. The sunscreen matrix 600 has a sun protection factor (“SPF”) rating based on the amount of sun blocking agents in the sunscreen matrix 600. The one or more sun blocking agents can include, but is not limited to, avobenzone, benzophenones, oxybenzone, dioxybenzone, sulisobenzone, mexoryl SX, also known as ecamsule, menthyl anthranilate, also known as meradimate, titanium dioxide, zinc oxide, and combinations thereof. Without being bound by theory, zinc oxide or titanium dioxide can be bound in the biocompatible matrix 600 and then bond with skin surface 603 by way of the silane. Chemical sunscreens, such as, for example, avobenzone, octinoxate, and octocrylene, among others, can be bound in the in the biocompatible matrix 600 and then bond with skin surface 603. Since it is a good solvent for both mineral and chemical sunscreens, alcohol can be employed as a co-medicant in the biocompatible matrix 600, which can also decrease drying times of the biocompatible matrix 600.

The sunscreen matrix 600 is configured to block a portion of UVA rays. In some aspects, the sunscreen matrix 600 is configured to block a portion of UVA rays and UVB rays. For example, full spectrum sunlight 640 is received by the sunscreen matrix 600, which reflects at least a portion of ultraviolet light 650 away from the skin surface 603.

Also illustrated in FIG. 6 is the repairing of skin by LWM-HA properties 630. Various properties 630 are transferred from the hydrogel 610 in the matrix 600 onto the skin surface 603 and the some of the properties 630 can be transported into the dermis 605.

The HA in the hydrogel 610 plays an important role in the normal epidermis. in the reepithelization process of skin due to several of its properties 630. These include being an integral part of the extracellular matrix of basal keratinocytes, which are major constituents of the epidermis; its free-radical scavenging function, and its role in keratinocyte proliferation and migration.

In normal skin, HA is found in relatively high concentrations in the basal layer of the epidermis where proliferating keratinocytes are found. CD44 is collocated with HA in the basal layer of epidermis where additionally it has been shown to be preferentially expressed on plasma membrane facing the HA-rich matrix pouches. Maintaining the extracellular space and providing an open, as well as hydrated, structure for the passage of nutrients are the main functions of HA in epidermis. Increasing HA concentration hydrates the skin and maintains the extracellular space in the skin, which improves the smoothness of the skin surface 603. In other words, increasing HA concentration in skin removes signs of aging from the skin surface 603.

The HA content increases in the presence of retinoic acid (vitamin A). The proposed effects of retinoic acid against skin photo-damage and photoaging may be correlated, at least in part, with an increase of skin HA content, giving rise to increased tissue hydration. It has been suggested that the free-radical scavenging property of HA contributes to protection against solar radiation, supporting the role of CD44 acting as a HA receptor in the epidermis.

Keratinocytes are the primary type of cell found in the epidermis, and form a barrier against environmental damage by heat, UV radiation, water loss, pathogenic bacteria, fungi, parasites, and viruses. HA also functions as a manipulator in the process of keratinocyte proliferation, which is essential in normal epidermal function, as well as during reepithelization in tissue repair. Increasing HA concentration improves keratinocyte proliferation in the skin and maintains the extracellular space in the skin, which improves the smoothness of the skin surface 603. In other words, increasing HA concentration in skin slows epidermal stem cell aging and increases keratinocyte proliferation in the dermis 605. HA-activated CD44 signaling can promote keratinocyte activities and improves abnormal epidermal functions.

Those of skill in the art can tune the hydrogel 410 to improve the moisturizing properties, which is discussed in, for example, Juncan, A. M, et al, “Advantages of Hyaluronic Acid and Its Combination with Other Bioactive Ingredients in Cosmeceuticals.” Molecules 2021, 26, 4429. For example, dry, scaly skin, such as that caused by atopic dermatitis, may be treated with lotion or another skin product containing sodium hyaluronate as its active ingredient.

HA is a common ingredient in skin care products. HA can facilitate the absorption of biomacromolecules, such as medicants, pharmaceuticals, and the like. HA can function like a nanocarrier to transport a medicant through the skin surface 603 and into the dermis 605. The effectiveness of HA as a microcarrier into the skin surface 603 depends on the hyaluronate formulation and skin health. In normal skin, LMW-HA enhances penetration of medicants into the dermis 605.

### Compositions for Treating Skin Conditions

Various embodiments provide compositions for treating skin conditions. A treatment composition can comprise an organosilane compound and a low molecular weight hyaluronic acid. In some embodiments, the treatment composition can comprise a quaternary ammonium organosilane compound, a low molecular weight hyaluronic acid, an organic acid, an alcohol, and water.

The treatment composition is an antimicrobial. In some embodiments, the treatment composition further comprises a medicant, as described herein. In some embodiments of the treatment composition, the medicant can be salicylic acid. In some configurations, the treatment composition can further comprise a fragrance. In some embodiments, the organosilane quaternary ammonium compound is octadecyldimethyl (3-triethoxysilylpropyl) ammonium chloride. The low molecular weight hyaluronic acid can be in a range from 5 kDa to 20 kDa.

In some embodiments, the quaternary ammonium organosilane compound is in a range from 0.1% to 1% of the weight of the composition. The low molecular weight hyaluronic acid is in a range from 0.01% to 2% of the weight of the composition. The organic acid is in a range from 0.01% to 2% of the weight of the composition. The alcohol is in a range from 0.01% to 5% of the weight of the composition. The water is in a range from 90% to 99% of the weight of the composition. In certain embodiments, the salicylic acid in the composition is in a range from 0.1% to 2% of the weight of the composition.

The treatment composition is typically a liquid at room temperature. The treatment composition is a liquid in a temperature range from 5° C. to 90° C. In some aspects of treating a skin condition, the treatment composition can be sprayed onto the skin surface of a user (patient) to form a coating over the skin surface.

In some embodiments, the organosilane quaternary ammonium compound crosslinks with the low molecular weight hyaluronic acid upon the composition being applied to a surface of skin.

Organosilane compounds are hybrid materials comprising various functional group connected to alternating silicon and carbon atoms as a backbone, which have versatile properties and a wide range of applications. In general, organosliane compounds have two reactive groups; organofunctional groups and a hydrolysable alkoxy. In various embodiments, an organosilane compounds can have the formula Rn SiX 4-n, where Rn is an organofunctional group, Si is silicon, X is a hydrolysable group, and n is 1, 2 or 3.

Hydrolysis and condensation are the two main reactions carried out by the molecules of organofunctional trialkoxy silanes. In hydrolysis, hydrolysable alkoxy groups are hydrolysed to silanols (Si—OH) and condensation takes place among silanols to form Si—O—Si (siloxane) structure. A siloxane is a type of chemical compound that consists of alternating silicon and oxygen atoms, with organic groups attached to the silicon atoms. Siloxanes are part of a larger family of compounds known as organosilicon compounds or organosiloxanes. Siloxanes have a unique structure that combines the inorganic nature of silicon-oxygen bonds (silicones) with organic functional groups attached to the silicon atoms.

Siloxanes can vary in their molecular structure and properties depending on the specific organic groups attached to the silicon atoms. These organic groups determine the characteristics and functionality of the siloxane compound.

Siloxanes, particularly cyclic siloxanes like cyclomethicone and cyclopentasiloxane, are used in many cosmetics and personal care products due to their smooth texture, spreadability, and volatility. Such siloxanes can act as emollients, conditioners, and carriers for other ingredients.

In various embodiments, the organosilane compound is a quaternary ammonium organosilane compound. In some embodiments, a quaternary ammonium organosilane compound can have the formula:

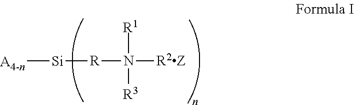

In Formula I, A is selected from —OR⁴, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. Where more than one A is present, each A is independently selected from the groups recited above or below.

R⁴ is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R is selected from substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene.

R¹, R², and R³ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

Z is selected from fluoride, chloride, bromide, iodide, tosylate, hydroxide, sulfate and phosphate. The symbol n is 1, 2 or 3.

In an exemplary implementation, each substituted alkyl, substituted heteroalkyl, substituted cycloalkyl, substituted heterocycloalkyl, substituted aryl, and substituted heteroaryl described herein as possible A, R¹, R², R³, and R⁴ moieties are substituted only with at least one substituent independently selected from —OH, unsubstituted (C₁-C₅)alkyl, unsubstituted 2 to 5 membered heteroalkyl, unsubstituted (C₅-C₇) membered cycloalkyl, unsubstituted 5 to 7 membered heterocycloalkyl, unsubstituted aryl, and unsubstituted heteroaryl. For example, where A is a substituted (C₁-C₁₀)alkyl, the substituted (C₁-C₁₀)alkyl is substituted only with at least one substituent independently selected from —OH, unsubstituted (C₁-C₅)alkyl, unsubstituted 2 to 5 membered heteroalkyl, unsubstituted (C₅-C₇) membered cycloalkyl, unsubstituted 5 to 7 membered heterocycloalkyl, unsubstituted aryl, and unsubstituted heteroaryl.

In other implementations, each substituted alkyl, substituted heteroalkyl, substituted cycloalkyl, substituted heterocycloalkyl, substituted aryl, and substituted heteroaryl described herein as possible A, R¹, R², R³, and R⁴ moieties are substituted only with at least one substituent independently selected from —OH, unsubstituted (C₁-C₅)alkyl, unsubstituted 2 to 5 membered heteroalkyl, unsubstituted (C₅-C₇) membered cycloalkyl, unsubstituted 5 to 7 membered heterocycloalkyl, unsubstituted aryl, and unsubstituted heteroaryl. In other implementations, each substituted alkyl, substituted heteroalkyl, substituted cycloalkyl, substituted heterocycloalkyl, substituted aryl, and substituted heteroaryl described herein as possible A, R¹, R², R³, and R⁴ moieties are substituted only with at least one substituent independently selected from —OH, unsubstituted (C₁-C₅)alkyl, unsubstituted (C₅-C₇) membered cycloalkyl, and unsubstituted phenyl. In yet other implementations, each substituted alkyl, substituted heteroalkyl, substituted cycloalkyl, substituted heterocycloalkyl, substituted aryl, and substituted heteroaryl described herein as possible A, R¹, R², R³, and R⁴ moieties are substituted only with at least one unsubstituted (C₁-C₃)alkyl.

In another exemplary embodiment, each substituted alkylene, substituted heteroalkylene, substituted cycloalkylene, substituted heterocycloalkylene, substituted arylene, and substituted heteroarylene described herein as possible R moieties are substituted only with at least one substituent independently selected from —OH, unsubstituted (C₁-C₅)alkyl, unsubstituted 2 to 5 membered heteroalkyl, unsubstituted (C₅-C₇) membered cycloalkyl, substituted 5 to 7 membered heterocycloalkyl, unsubstituted aryl, and unsubstituted heteroaryl.

In various implementations, each substituted alkylene, substituted heteroalkylene, substituted cycloalkylene, substituted heterocycloalkylene, substituted arylene, and substituted heteroarylene described herein as possible R moieties are substituted only with at least one substituent independently selected from —OH, unsubstituted (C₁-C₅)alkyl, unsubstituted 2 to 5 membered heteroalkyl, unsubstituted (C₅-C₇) membered cycloalkyl, unsubstituted 5 to 7 membered heterocycloalkyl, unsubstituted aryl, and unsubstituted heteroaryl. In other implementations, each substituted alkylene, substituted heteroalkylene, substituted cycloalkylene, substituted heterocycloalkylene, substituted arylene, and substituted heteroarylene described herein as possible R moieties are substituted only with at least one substituent independently selected from —OH, unsubstituted (C₁-C₅)alkyl, unsubstituted (C₅-C₇) membered cycloalkyl, and unsubstituted phenyl. In yet other implementations, each substituted alkylene, substituted heteroalkylene, substituted cycloalkylene, substituted heterocycloalkylene, substituted arylene, and substituted heteroarylene described herein as possible R moieties are substituted only with at least one unsubstituted (C₁-C₃)alkyl.

A may be selected from —OR⁴, substituted or unsubstituted (C₁-C₁₀)alkyl, substituted or unsubstituted 2 to 12 membered heteroalkyl, substituted or unsubstituted (C₅-C₇) cycloalkyl, substituted or unsubstituted 5 to 7 membered heterocycloalkyl, substituted or unsubstituted aryl, and substituted or unsubstituted heteroaryl. R⁴ may be selected from hydrogen, substituted or unsubstituted (C₁-C₁₀)alkyl, substituted or unsubstituted 2 to 10 membered heteroalkyl, substituted or unsubstituted (C₅-C₇) cycloalkyl, substituted or unsubstituted 5 to 7 membered heterocycloalkyl, substituted or unsubstituted aryl, and substituted or unsubstituted heteroaryl.

In some implementations, A is selected from —OR⁴, unsubstituted (C₁-C₁₀)alkyl, unsubstituted 2 to 12 membered heteroalkyl, unsubstituted (C₅-C₇) cycloalkyl, unsubstituted 5 to 7 membered heterocycloalkyl, unsubstituted aryl, and unsubstituted heteroaryl. In other implementations, A is selected from —OR⁴, unsubstituted (C₁-C₁₀)alkyl, unsubstituted 3 to 12 membered alkylether, unsubstituted (C₅-C₇) cycloalkyl, and unsubstituted phenyl.

A may also be selected from —OR⁴, unsubstituted (C₁-C₄)alkyl, unsubstituted 3 to 8 membered alkylether, unsubstituted (C₅-C₇) cycloalkyl, and unsubstituted phenyl. Alternatively, A is selected from —OR⁴, unsubstituted (C₁-C₄)alkyl, and unsubstituted 3 to 8 membered alkylether.

R⁴ may be selected from hydrogen, unsubstituted (C₁-C₁₀)alkyl, unsubstituted 2 to 12 membered heteroalkyl, unsubstituted (C₅-C₇) cycloalkyl, unsubstituted 5 to 7 membered heterocycloalkyl, unsubstituted aryl, and unsubstituted heteroaryl.

In some implementations, R⁴ is selected from hydrogen, unsubstituted (C₁-C₁₀)alkyl, unsubstituted 3 to 12 membered alkylether, unsubstituted (C₅-C₇) cycloalkyl, and unsubstituted phenyl. In a related embodiment, R⁴ is selected from hydrogen, unsubstituted (C₁-C₈)alkyl, unsubstituted 3 to 8 membered alkyl ether, unsubstituted (C₅-C₇) cycloalkyl, and unsubstituted phenyl. Alternatively, R⁴ is selected from hydrogen, unsubstituted (C₁-C₈)alkyl, and unsubstituted 3 to 8 membered alkyl ether.

R⁴ may also be selected from phenyl, methylphenyl, substituted or unsubstituted (C₁-C₈)alkyl, and —(CH₂)ₓ—O—(CH₂)_{y}CH₃. X and y are integers independently selected from 1 to 10.

R may be selected from substituted or unsubstituted (C₁-C₁₀)alkylene, substituted or unsubstituted 2 to 10 membered heteroalkylene, substituted or unsubstituted (C₅-C₇) cycloalkylene, substituted or unsubstituted 2 to 7 membered heterocycloalkylene, substituted or unsubstituted arylene, and substituted or unsubstituted heteroarylene.

In various implementations, R is a member selected from unsubstituted (C₁-C₁₀)alkylene, unsubstituted 2 to 10 membered heteroalkylene, unsubstituted (C₅-C₇) cycloalkylene, unsubstituted 5 to 7 membered heterocycloalkylene, unsubstituted arylene, and unsubstituted heteroarylene. R may also be unsubstituted (C₁-C₁₀)alkylene.

R¹, R², and R³ may be selected from hydrogen, substituted or unsubstituted (C₁-C₂₀)alkyl, substituted or unsubstituted 2 to 20 membered heteroalkyl, substituted or unsubstituted (C₅-C₇) cycloalkyl, substituted or unsubstituted 5 to 7 membered heterocycloalkyl, substituted or unsubstituted aryl, and substituted or unsubstituted heteroaryl.

In some implementations, R¹, R², and R³ are independently selected from hydrogen, unsubstituted (C₁-C₂₀)alkyl, hydroxy-substituted (C₁-C₂₀)alkyl, amine-substituted (C₁-C₂₀)alkyl, unsubstituted 2 to 20 membered heteroalkyl, unsubstituted (C₅-C₇) cycloalkyl, unsubstituted 5 to 7 membered heterocycloalkyl, unsubstituted aryl, and unsubstituted heteroaryl. In a related embodiment, R¹, R², and R³ are independently selected from hydrogen, unsubstituted (C₁-C₂₀)alkyl, unsubstituted alkylether, hydroxy-substituted (C₁-C₂₀)alkyl, amine-substituted (C₁-C₂₀)alkyl, unsubstituted (C₅-C₇) cycloalkyl, and unsubstituted phenyl.

R¹, R², and R³ may also be selected from hydrogen, unsubstituted (C₁-C₂₀)alkyl, unsubstituted alkylether, hydroxy-substituted (C₁-C₂₀)alkyl, amine-substituted (C₁-C₂₀)alkyl, unsubstituted (C₅-C₇) cycloalkyl, and unsubstituted phenyl. Alternatively, R¹, R², and R³ are selected from hydrogen, unsubstituted (C₁-C₂₀)alkyl, unsubstituted alkylether, hydroxy-substituted (C₁-C₂₀)alkyl, and amine-substituted (C₁-C₂₀)alkyl.

In other exemplary embodiments, R¹, R², and R³ are independently selected from —(CH₂)_{q}OCH₃, —(CH₂)_{q}OH, —(CH₂)ₜO(CH₂)_{q}CH₃, —(CH₂)_{q}NHCH₃, —(CH₂)_{q}NH₂, —(CH₂)_{q}N(CH₃)₂ and —(CH₂)_{q}NH₂ (CH₂)ₜCH₃, in which q and t are integers independently selected from 0 to 10. R¹, R², and R³ may also be independently selected from the group consisting of —CH₂CH₂OCH₃ and —CH₂CH₂OCH₂CH₂CH₃. Alternatively, R¹, R², and R³ may also be independently selected from —CH₂CH₂OH and —CH₂CH₂CH₂CH(OH) CH₃. R¹, R², and R³ may also be independently selected from —CH₂CH₂NH₂ and —CH₂CH₂N(CH₃)₂. Finally, R¹, R², and R³ may be members independently selected from methyl, octadecyl, didecyl, and tetradecyl.

In an exemplary embodiment, the quaternary ammonium organosilane reagent is selected from (CH₃O)₃Si(CH₂)₃N⁺ (CH₃)₂ (C₁₈H₃₇)(Cl⁻); (CH₃CH₂O)₃Si(CH₂)₃N⁺ (CH₃)₂ (C₁₈H₃₇)(Cl⁻); (CH₃O)₃Si(CH₂)₃N⁺ (CH₃)₂ (C₁₈H₃₇) (Br⁻); (CH₃O)₃Si(CH₂)₃N⁺ (C₁₀H₂₁)₂ (CH₃)(Cl⁻); (CH₃O)₃Si(CH₂)₃N⁺ (CH₃)₂ (C₁₄H₂₉) (Cl⁻); (CH₃O)₃Si(CH₂)₃N⁺ (CH₃)₂ (C₁₄H₂₉)(Br⁻); and (CH₃O)₃Si(CH₂)₃N⁺ (CH₃)₂ (C₁₆H₃₃)(Cl⁻). In a related embodiment, the quaternary ammonium organosilane selected reagent is from 3-(trimethoxysilyl)propyldimethyloctadecyl ammonium chloride, 3-(trimethoxysilyl)propyldidecylmethyl ammonium chloride, and 3-(trimethoxysilyl)propyldimethyltetradecyl ammonium chloride.

In some embodiments, the organosilane quaternary ammonium compound can be selected from the group consisting of: of 1-Octadecanaminium, N,N-dimethyl-N-(3-(trimethoxysilyl) propyl)-, chloride; 3-(Trimethoxysilyl) propyl dimethyl octadecyl ammonium chloride; 3-(Trimethoxysilyl) propyldimethyloctadecylammonium; Dimethylocadecyl (3-(Trimethoxysilyl) propyl) ammonium chloride; Dow Corning 5700; N,N-Dimethyl-N-(3-(Trimethoxysilyl) Propyl)-1-Octadecanaminium Chloride; Octadecyldimethyl (3-(trimethoxysilyl) propyl) ammonium chloride, and combinations thereof.

In another exemplary embodiment, the quaternary ammonium organosilane contains an ammonium halide and a hydrolyzable alkoxy group bonded to silicon.

In some embodiments, the organosilane quaternary ammonium compound is octadecyldimethyl (3-triethoxysilylpropyl) ammonium chloride. In some embodiments, the organosilane quaternary ammonium compound is Octadecyldimethyl (3-trihydroxysilylpropyl) Ammonium Chloride (CAS #62117-57-1). In some embodiments, the organosilane quaternary ammonium compound is 1-Octadecanaminium, N,N-dimethyl-N-(3-(trimethoxysilyl) propyl)-chloride (CAS #27668-52-6).

Hyaluronic acid (“HA”), also known as hyaluronan, is a substance naturally found in the human body, particularly in the skin, connective tissues, and eyes. HA is an anionic, non-sulfated glycosaminoglycan HA is a polymer of disaccharides, which are composed of D-glucuronic acid and N-acetyl-D-glucosamine, linked via alternating β-(1→4) and β-(1→3) glycosidic bonds.

Polymers of HA can range in size from 4 kDa to 60,000 kDa, Typically, HA is categorized into high molecular weight (“HMW”) HA and low molecular weight (“LMW”) HA. The HMW HA is a very large molecule and the natural form of HA. The LMW-HA has a molecular weight in the range from 5 kDa to 100 kDa. In some embodiments, the LMW-HA has a molecular weight in the range from 5 kDa to 20 kDa. In some embodiments, the LMW-HA has a molecular weight in the range from 8 kDa to 15 kDa.

As known to those of skill in the art, HA can be cross-linked to modify its properties for various applications. Cross-linking is a process in which the HA molecules are chemically bonded together to form a three-dimensional network or gel-like structure. This cross-linking can alter the viscosity, stability, and longevity of hyaluronic acid-based products.

Cross-linked HA is commonly used in the field of aesthetic medicine for dermal fillers. By cross-linking the HA molecules, the resulting gel has a higher viscosity and provides longer-lasting results when injected into the skin to add volume, reduce wrinkles, or enhance facial contours. The degree of cross-linking can be adjusted to achieve different textures and properties suitable for specific purposes. For example, some cross-linked HA products can be designed to be more durable and provide structural support, while others can be formulated to be softer and more flexible for delicate areas or fine lines.

As known to those of skill in the art, HA can be cross-linked with various polymers to create hybrid materials with unique properties. Cross-linking HA with polymers can enhance its stability, mechanical strength, and control the release of drugs or bioactive molecules. Those of skill in the art can tune the hydrogel in a biocompatible matrix to improve the release of a medicant (such as, a drug or a bioactive molecule), which is discussed in, for example, Lee et. al. One-pot synthesis of silane-modified hyaluronic acid hydrogels for effective antibacterial drug delivery via sol-gel stabilization.”, Colloids and Surfaces 174 2019, 308-315.

One common approach is to cross-link HA with polyethylene glycol (PEG) to form a hydrogel. The resulting hybrid hydrogel combines the biocompatibility and water-absorbing properties of hyaluronic acid with the mechanical strength and stability provided by PEG. This type of cross-linked hyaluronic acid hydrogel has been extensively studied for tissue engineering, drug delivery systems, and wound healing applications. Other polymers that can be used for cross-linking with HA include poly(vinyl alcohol), poly(N-isopropylacrylamide), and poly(lactic-co-glycolic acid).

Based on the unexpected results discussed herein, this is the first time that crosslinking HA with an organosilane has been reported. Furthermore, this is the first time that crosslinking HA with a quaternary ammonium organosilane has been reported.

The specific cross-linking method and polymer choice can influence the mechanical, chemical, and biological properties of the resulting material. The choice of cross-linking strategy and polymer must be carefully considered to ensure biocompatibility, stability, and functionality for the intended application. The development of cross-linked HA-polymer hybrids often involves thorough research and optimization to achieve the desired properties and performance.

As described above, an organic acid can be a component in the various formulations of the treatment composition. Organic acid is a type of organic compound that contains one or more carboxyl functional groups (—COOH) attached to a carbon atom. This functional group consists of a carbonyl group (C═O) and a hydroxyl group (—OH) bonded to the same carbon atom. In some embodiments of the treatment composition, the organic acid can be selected from the group consisting of acetic acid, carboxylic acid, citric acid, formic acid, lactic acid, malic acid, and combinations thereof.

A weak organic acid is an organic acid that does not completely dissociate into ions when dissolved in water. Instead, it partially ionizes, meaning only a fraction of the acid molecules donate a proton (H+) to water molecules. As a result, the concentration of hydronium ions (H3O+) in the solution is relatively low. Weak organic acids have a characteristic equilibrium between their undissociated form and their dissociated ions in an aqueous solution, which is measured as the acid disassociation constant or pKa.

In some embodiments of the treatment composition, the organic acid is a weak acid. In some embodiments of the treatment composition, the organic acid has a pKa in a range from 2.5 to 6.5. In some embodiments of the treatment composition, the organic acid can be selected from a group of weak acids consisting of acetic acid, acetylsalicylic acid, benzoic acid, butyric acid, carboxylic acid, citric acid, formic acid, fumaric acid, glycolic acid, lactic acid, malic acid, oxalic acid, phosphoric acid, propionic acid, succinic acid, tartaric acid, uric acid, and combinations thereof.

The concentration of the organic acid in the treatment composition can be in a range from 0.01% to 2% of the weight of the composition. In some formulations, the concentration of the organic acid in the treatment composition can be in a range from 0.04% to 0.07% of the weight of the composition.

A medicant is a medicinal substance that is effective in treating a medical condition. As discussed herein, the treatment composition can comprise one or more medicants. For example, the medicant can be benzoyl peroxide, salicylic acid, a topical retinoid, an antibiotic, one of more vitamins, a hormones, isotretinoin, an anti-inflammatory and combinations thereof. In some embodiments, the treatment composition comprises an effective amount of an medicant for the treatment of a skin condition.

In some embodiments, alcohol can be a medicant. In some embodiments, the treatment composition comprises alcohol. The alcohol is selected from the group consisting of methanol, ethanol, propanol, isopropyl, and combinations thereof.

The concentration of the alcohol in the treatment composition can be in a range from 0.01% to 80% of the weight of the composition. In some formulations, the concentration of the alcohol in the treatment composition can be in a range from in a range from 0.1% to 0.1% of the weight of the composition.

In some embodiments, the treatment composition can also comprise other components, which can include, but is not limited to, compounds listed below, such as a diol, an emulsifier, a fragrance, a fruit extract, a vegetable extract, a SPF material, an emollient, an astringent, a humectant, and other such compounds known to those of skill in the art of cosmetic products.

A diol can be a component in the various formulations of the treatment composition. A diol is an organic compound that contains two hydroxyl (—OH) functional groups. Without being bound by theory, the presence of multiple hydroxyl groups in diols provides them with reactivity, which enables diols to participate in polymer condensation reactions.

The diol can be selected from the group consisting of ethylene glycol, glycerol, diethylene glycol, polyethylene glycol, 1,3-propanediol, 1,4-butanediol, 1,5-pentanediol, 1,6-hexanediol, 1,10-decanediol, and combinations thereof. The concentration of the diol in the treatment composition can be in a range from 0.1% to 5% of the weight of the composition. In some formulations, the concentration of the diol in the treatment composition can be in in a range from 1% to 2% of the weight of the composition.

An emulsifier can be a component in the various formulations of the treatment composition. An emulsifier, also known as an emulsifying agent or emulsion stabilizer, is a substance that helps to form and stabilize emulsions. In an emulsion, the emulsifier reduces the interfacial tension between the two immiscible liquids, allowing them to mix together and form a stable mixture. The emulsifier is a molecule with both hydrophilic and hydrophobic properties. The hydrophilic part of the emulsifier molecule interacts with water, while the hydrophobic part interacts with the oil or other immiscible liquid. Emulsifiers play a crucial role in cosmetic products, where oil-in-water or water-in-oil emulsions are commonly used. Emulsifiers create stable and homogeneous formulations, which enable the proper dispersion and stability of a cosmetic product, and the desired texture, appearance, and sensory properties of the cosmetic product.

In some embodiments, the treatment composition comprises an emulsifier. The emulsifier is selected from the group consisting of lauryl glucoside, mineral oil, emulsifying wax, hydrogenated castor oil, cetearyl alcohol, glyceryl stearate, polyethylene glycol, distearyldimonium chloride, and combinations thereof. The concentration of the emulsifier in the treatment composition can be in a range from 0.1% to 10% of the weight of the composition. In some formulations, the concentration of the emulsifier in the treatment composition can be in in a range from 3% to 4% of the weight of the composition. In some formulations, the emulsifier is PEG.

A fragrance can be a component in the various formulations of the treatment composition. Fragrances are widely used in cosmetic products to enhance their appeal, provide a pleasant scent, and create a positive sensory experience for consumers. In some embodiments, the treatment composition comprises a fragrance. The fragrance is selected from the group consisting of water-soluble fragrance compounds.

The concentration of the fragrance in the treatment composition can be in be in a range from 0.01% to 1% of the weight of the composition. In some formulations, the concentration of the fragrance in the treatment composition can be in a range from 0.01% to 0.02%.

Exemplary SPF materials are discussed in the description of FIG. 6.

### Methods of Making the Treatment Composition

An exemplary method of making the treatment composition can include multiple steps.

Step 1, The LMW-HA solution can be prepared as follows:

Preparing a 1% aqueous solution of LMW-HA. Sodium hyaluronate is weighted out and added to DI water. The mixture is stirred via spin bar for approximately 15 minutes to an hour until all powder has been dissolved in solution.

Step 2. The silane solution can be prepared as follows:

Preparing a 0.41% solution of Octadecyldimethyl (3-trihydroxysilylpropyl) Ammonium Chloride. (CAS #62117-57-1).

Step 3. Combining the LMW-HA solution and silane solution with other components.

Preparing the treatment composition. Heating (below the boiling point of water) the LMW-HA solution over a water bath, mixing in up to 1% of an organic acid, mixing in up to 5% of a medicant, and then mixing in the silane solution. Continue to mix the treatment composition until the solution is clear.

Step 4. The treatment composition is stable as a liquid in a temperature range from 5° C. to 90° C. The treatment composition can be packaged into a container with an atomizer configured to spray an aerosol of the treatment composition onto a treatment area of a skin surface of a patient.

A second exemplary method of making the treatment composition can include multiple steps.

Step 1. Weight out and combine salicylic acid, propanediol 1,3, and PEG 60 hydrogenated castor oil. Once all components are combined, place the vessel to a hot water bath. Continue to heat (below the boiling point of water) with intermittent stirring until all components are dissolved and solution is translucent with no solids present. Set vessel aside for addition this component mixture in Step 3.

Step 2. Add DI water to a second vessel and mix at medium speed. Add 1% stock solution of the organic acid and continue to stir for about 3 minutes. Slowly add the Octadecyldimethyl (3-Triethoxysilylpropyl) ammonium chloride (CAS #62117-57-1). When addition of the silane compound is completed continue to stir for about 3 minutes. Then add a fruit extract while continuing the mixing of the aqueous solution. Add 1% stock solution of LMW-HA (CAS #9067-32-7) and continue to stir for about 3 minutes. Add the fragrance and triethanolamine and continue to stir for about 5 minutes until solution is clear and homogenous.

Step 3. While continuing to slowly stir the aqueous solution of Step 2, add the component mixture of Step 1. After addition of this component mixture, continue to stir for about 20 minutes until the treatment composition solution is clear and homogenous.

Step 4. The pH range of the treatment composition solution is between 3.7-3.9. Add triethanolamine to adjust pH of the treatment composition solution, as necessary.

Step 5. The treatment composition solution is stable as a liquid in a temperature range from 5° C. to 90° C. The treatment composition solution can be packaged into a container with an atomizer configured to spray an aerosol of the treatment composition onto a treatment area of a skin surface of a patient.

The resulting treatment composition solution can comprise LMW-HA (about 1%), 1-octadecanaminium, N,N-dimethyl-N-[3-(trihydroxysilyl) propyl]ammonium chloride (>1%), salicylic acid (about 0.5%), propanediol 1,3 (about 1.5%), watermelon extract (about 0.5%), an organic acid (>0.5%), and PEG 60 hydrogenated castor oil (about 4%), and cucumber fragrance (about 0.1%), in water (at least 90%).

### Product by Process

A skin treatment product is made by a process of making the treatment composition, as discussed above. In another example, the a process of making the treatment composition includes the steps in the second exemplary method of making the treatment composition In one example, the a process of making the treatment composition includes the steps in the exemplary method of making the treatment composition,

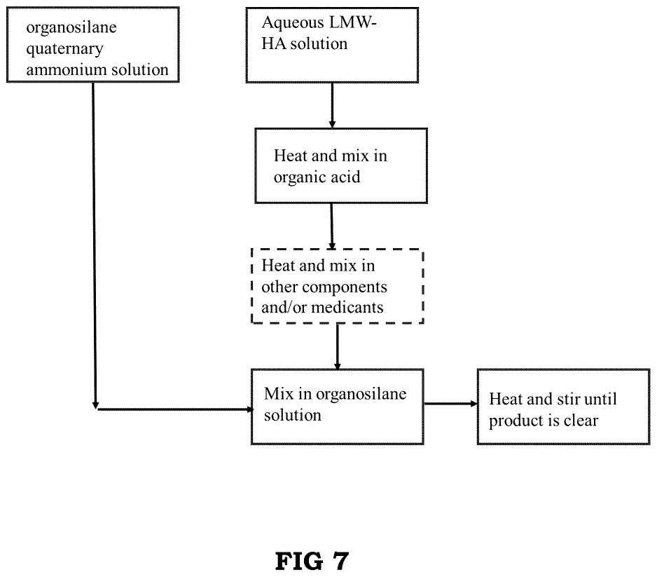

With reference to FIG. 7, an exemplary product by process illustrated in a flow chart. An aqueous LMW-HA solution is heated, and organic acid is mixed into the solution. The temperature of the heated solution is below the boiling point of water. Optionally, other components and/or medicants can be mixed into the solution while the solution is heated. Examples of other components include fragrances, diols, emulsifiers, an emollient, an astringent, a humectant, and any other cosmetic component, as selected by one of skill in the art. Examples of a medicant include benzoyl peroxide, salicylic acid, topical retinoids, alcohol, antibiotics, an anti-inflammatory, hormones, isotretinoin, and combinations thereof.

The product is created by mixing the organosilane quaternary ammonium solution into the above combined solution, which is still heated. The product is stirred, while still being heated, until the solution is clear. The product is a skin treatment composition, as described herein.

A second product by process is formed when the skin treatment composition is applied to a skin surface. The second product by process is the biocompatible matrix, as described herein.

The aqueous formulations containing LMW-HA and a oligomeric water soluble organosilane species having a silicon-bonded quaternary ammonium halide functionality and hydrolyzable alkoxy groups and for their application to human skin wherein the oligomeric species react with the HA and on the skin to provide a product that is a polymeric biocompatible matrix which is substantive and bound to the skin.

In various embodiments provide a skin treatment system. A skin treatment system can include an aqueous treatment composition comprising hyaluronic acid, at least one silane, an organic acid, and water; a container configured to hold the liquid composition; and a spray device coupled to the container and in communication with the liquid.

The aqueous treatment composition can be the treatment composition, as described above. For certain skin conditions, the aqueous treatment composition can comprise an organosilane quaternary ammonium compound, a low molecular weight hyaluronic acid, a medicant, an organic acid, and water. In some embodiments, the composition comprises salicylic acid. The aqueous treatment composition is a liquid at room temperature.

In some embodiments, the organosilane quaternary ammonium compound is in a range from 0.1% to 1% of the weight of the aqueous treatment composition. The low molecular weight hyaluronic acid is in a range from 0.01% to 2% of the weight of the aqueous treatment composition. The organic acid is in a range from 0.01% to 2% of the weight of the aqueous treatment composition. The alcohol is in a range from 0.01% to 5% of the weight of the aqueous treatment composition. The water is in a range from 90% to 99% of the weight of the aqueous treatment composition. The salicylic acid in the aqueous treatment composition is in a range from 0.1% to 2% of the weight of the composition. The liquid composition is a liquid in a temperature range from 5° C. to 90° C. In some embodiments, the aqueous treatment composition can comprise 1-Octadecanaminium, N,N-dimethyl-N-[3-(trihydroxysilyl) propyl]. In some embodiments, the aqueous treatment composition can comprise octadecyldimethyl (3-triethoxysilylpropyl) ammonium chloride.

The at least one silane in the skin treatment system is octadecyldimethyl (3-triethoxysilylpropyl) ammonium chloride in a weight percentage range from 0.1% to 1%; and the hyaluronic acid has a molecular weight in the range from 5 kDa to 20 kDa and is in a weight percentage range from 0.01% to 2%.

In some embodiments, the organosilane quaternary ammonium compound crosslinks with the low molecular weight hyaluronic acid upon the liquid composition being applied to a surface of skin.

A topically applicable pharmaceutical spray solution composition useful for the treatment of wounds consisting essentially of an effective amount of salicylic acid of 0.5% by weight thereof, formulated into a topically applicable, pharmaceutically acceptable spray solution medium comprising hyaluronic acid, at least one silane, an organic acid, and water.

In some examples of the topically applicable spray solution composition, the spray solution medium further comprises at least one ingredient selected from the group consisting of humectant agent, pro-penetrant agent, propylene glycol, polyethylene glycol, PEG400, a lipophilic substance, silicone fatty substances, non-siliconed fatty substances, and mixtures thereof.

In some examples of the topically applicable spray solution composition, the spray solution medium comprises: LMW-HA, 1-octadecanaminium,N,N-dimethyl-N-(3-(trimethoxysilyl) propyl-chloride (less than 1%), ethanol, and a cucumber fragrance.

In some examples, the topically applicable spray solution composition further comprises one or more of a medicant, an emollient, an astringent, a humectant, an antibiotic, and an anti-inflammatory.

In some examples of the topically applicable spray solution composition, the spray solution medium comprises at least one inert additive selected from the group consisting of: wetting agents; flavor enhancers; preservatives; stabilizers; moisture regulators; pH regulators; osmotic pressure modifiers; emulsifiers; antioxidants; and combinations thereof.

Various embodiments provide a skin treatment system. A skin treatment system can include an aqueous treatment composition comprising hyaluronic acid, at least one silane, an organic acid, and water; a container configured to hold the liquid composition; and a delivery device in communication with the liquid.

Examples of a delivery device include, but are not limited to, atomizers, facial pads, wipes, towelettes, and roller balls. In various embodiments, a surface of the delivery device is a non-organic material. For facial pads, wipes, and towelettes, the delivery device comprises a synthetic fiber substrate. Examples of synthetic fibers include polypropylene, polyester, or rayon. Constructing a synthetic fiber substrate as a delivery device for a liquid is well known to those of skill in the art. Since the aqueous treatment composition crosslinks upon application to an organic surface, a delivery device is not effective with such an organic surface.

If the substrates is made of inorganic polymers and the facial pads, wipes, or towelettes are stored in a container with excess aqueous treatment composition, the aqueous treatment composition will remain active and monomeric until it is applied to the skin surface. For example, shelf-life tests were conducted with polypropylene towelettes saturated in excess aqueous treatment composition stored in a resealable container. Over a period of a year, bromophenol blue testing was conducted on the aqueous treatment composition that was extracted from the towelette. After one year of the shelf-life tests, the anti-microbial activity of the aqueous treatment composition on the towelette at the beginning of the tests was equivalent to the anti-microbial activity of the aqueous treatment composition on the towelette at the end of the tests. For the shelf-life test, the aqueous treatment composition was an treatment composition, as described herein.

In some embodiments, the delivery device is a roller ball, which is coupled to the container configured to hold the aqueous treatment composition and the roller ball is in communication with the aqueous treatment composition. Examples of a roller ball substrate include, but are not limited to, inorganic polymers, stainless steel, aluminum, glass, and combinations thereof. These exemplary substrate materials will not react with the aqueous treatment composition. Accordingly, the organosilane quaternary ammonium compound and the low molecular weight hyaluronic acid will not polymerize on these exemplary substrate materials.

In some embodiments, the delivery device is a pad, which can be configured to adhere to the surface of the skin. For example, the pad is an aqueous treatment impregnated hydrocolloid patch for spot treatment of a wound.

The aqueous treatment composition can be any treatment composition, as described above. For certain skin conditions, the aqueous treatment composition can comprise an organosilane quaternary ammonium compound, a low molecular weight hyaluronic acid, a medicant, an organic acid, and water. In some embodiments, the composition comprises salicylic acid. The aqueous treatment composition is a liquid at room temperature.

A cutting-edge method to produce an antimicrobial hyaluronic hydrogel with zero toxicity. This innovative process involves the fabrication of hyaluronan biopolymer hydrogel films using a rapid and cost-effective technique.

Hyaluronan infused antimicrobial bandages. Bandages that have a biodegradable Hyaluronic acid matrix throughout the non-woven structure for the purpose of providing wounds with a epithelialization scaffold for cellular colonization and the possibility of lowering infection.

This approach enables seamless integration into existing bandage materials like non-woven fabrics. The technique also teaches the formation of advanced hyaluronan hydrogel films that can be tailored for multiple applications.

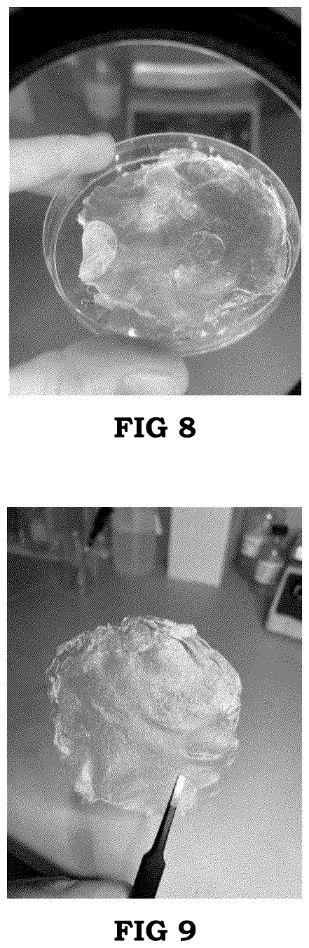

Moving to FIGS. 8 and 9, an example of a HMW Hyaluronan hydrogel sheet is illustrated. As discussed herein, the HMW HA is a large molecule with the hydroxylated subunits being repeated many times, which causes the HA to polymerize in a solution with a silane concentration ranging from 0.1%-5% almost immediately upon contact.

This polymerization can yield a biocompatible sheet, as illustrated in FIGS. 8 and 9. In some embodiments, a wound dressing device comprises a biocompatible sheet.

An exemplary biocompatible sheet was created with 500 kDa-785 kDa HA (Penta Manufacturing) and a siloxane using the Sol gel method. Another exemplary biocompatible sheet was created with 800-1500 kDa HA (makingcosmetics.com) and a siloxane using the Sol gel method.

A biocompatible sheet for wound care were prepared as follows:
1. The process starts with preparation of a 1% aqueous solution of HMW Hyaluronic acid. Sodium hyaluronate is weighed out and added to DI water. The mixture is stirred via spin bar for approx. 8 hours until all powder has been dissolved in solution.
2. The next step is to prepare a 0.71% solution of Octadecyldimethyl (3-trihydroxysilylpropyl) Ammonium Chloride. (CAS #62117-57-1 was utilized for this step).
3. A SOL-Gel method is used to crosslink and form an antimicrobial silsesquioxane HA matrix from the two materials.
4. Samples were air dried approx. 48 hr.

An example of general preparation of a biocompatible sheet infused withnon wovens:
1. Prepare a 1% solution of HMW sodium hyaluronate.
2. Prepare a 1% solution of Octadecyldimethyl (3-trihydroxysilylpropyl) Ammonium Chloride. (CAS #62117-57-1 was utilized for this step).
3. Weight out the desired amount of sodium hyaluronate to be added to the non-woven sample.
4. Add the Sodium hyaluronate to the non-woven until it has up taken and been saturated by the desired amount.
5. Once the sample has absorbed the HA the sample is added to the 1% SiQAC solution for approx. 2 hours
6. Sample is removed and air dried for 48 hours.

In some embodiments, active agents are incorporated directly into micro-cavities of the matrix of the biocompatible sheet. The agents may be incorporated by absorption of agents by the sheet and preferably by incorporation during the polymerization of the sheet. It is theorized that the release of the active agents may be controlled via manipulation of concentration parameters, movement of water through the sheet and the degree of cross linking in the sheet. In some embodiments, the wound dressings comprise a stranded configuration, wherein the strands extend from at least one common region and the strands themselves comprise a polymer matrix.

### EXAMPLES OF WOUND CARE

### Example 1

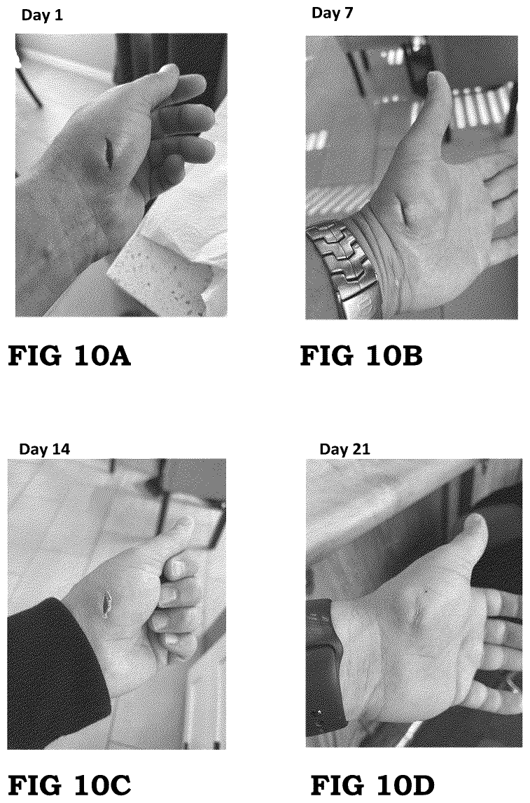

With reference to FIGS. 10A-10D, an example of wound care is illustrated. Subject had a laceration of approximately 17 mm long in the palm of his hand caused by a puncture with a box cutter, as illustrated in FIG. 10A. Subject stopped the bleeding with pressure for 15 min. Once the bleeding stopped, the wound was washed with soap & water then lightly wrapped with gauze wrap.

A method for wound care in an wound zone includes the following steps: administering a liquid composition onto the wound zone in a skin surface; crosslinking LMW-HA and 1-octadecanaminium, N,N-dimethyl-N-[3-(trihydroxysilyl) propyl] chloride in the liquid solution to create a biocompatible matrix; bonding a thin layer of the biocompatible matrix onto the skin surface over the wound zone. The liquid composition comprises a solution of LMW-HA (about 1%), 1-octadecanaminium,N,N-dimethyl-N-[3-(trihydroxysilyl) propyl]ammonium Chloride (>1%), in water (at least 90%).

Autocondensation occurs across the surface of skin to produce the biocompatible matrix, which is a 3-dimensional, cross-linked polymers being on the average of 10-20 molecules thick. Molecules that have not bonded with the 1-octadecanaminium, N,N-dimethyl-N-[3-(trihydroxysilyl) propyl] are free to pass from the biocompatible matrix onto the skin surface and into the dermis. In these examples, medicants, growth factors, and other healing components, discussed herein, can move from the biocompatible matrix into the wound zone.

Day 2-8: Subject sprayed wound with two to three pumps of an example of the treatment composition (WCP #32) in the morning, applied a butterfly bandage, and wrapped with gauze. In the afternoon, subject removed gauze and butterfly bandage, washed hands with soap and water, then dried repeated application. Subject sprayed the wound with two to three pumps of the treatment composition, applied a butterfly bandage and covered with gauze wrap. The healing process on Day 7 is illustrated in FIG. 10B.

Day 9-21: Subject applied two to three pumps of the treatment composition to the wound site twice daily without applying the butterfly bandage or the gauze wrap. Subject left wound uncovered and open to the environment. The healing process on Day 14 is illustrated in FIG. 10C. The wound is completely healed on Day 21, as illustrated in FIG. 10D.

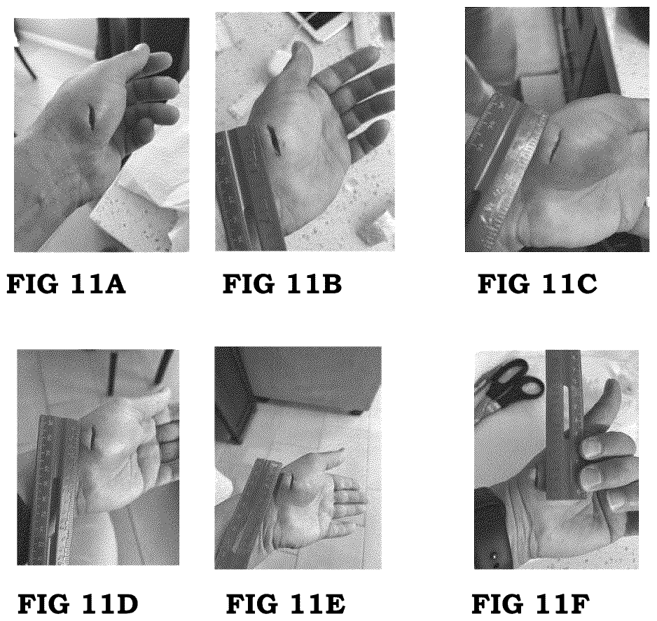

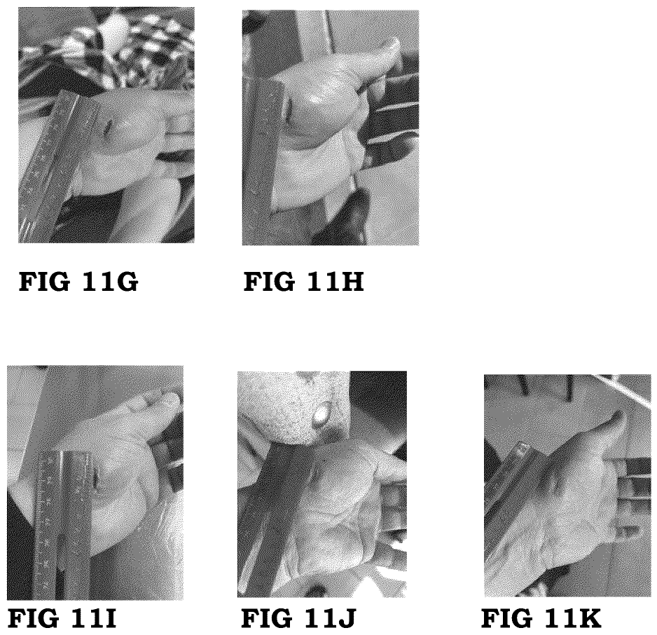

FIGS. 11A-11K illustrate the same example, as above, however, a ruler is included in the figures to understand the scale of the wound. The healing process on Day 1 is illustrated in FIG. 11A. The healing process at Day 2 is illustrated in FIG. 11B. The healing process on Day 3 is illustrated in FIG. 11C. The healing process at Day 4 is illustrated in FIG. 11D. The healing process at Day 6 is illustrated in FIG. 11E. The healing process on Day 7 is illustrated in FIG. 11F. The healing process on Day 10 is illustrated in FIG. 11G. The healing process on Day 11 is illustrated in FIG. 11H. The healing process on Day 12 is illustrated in FIG. 11I. The healing process on Day 19 is illustrated in FIG. 11J. The healing process on Day 21 is illustrated in FIG. 11K.

### Example 2

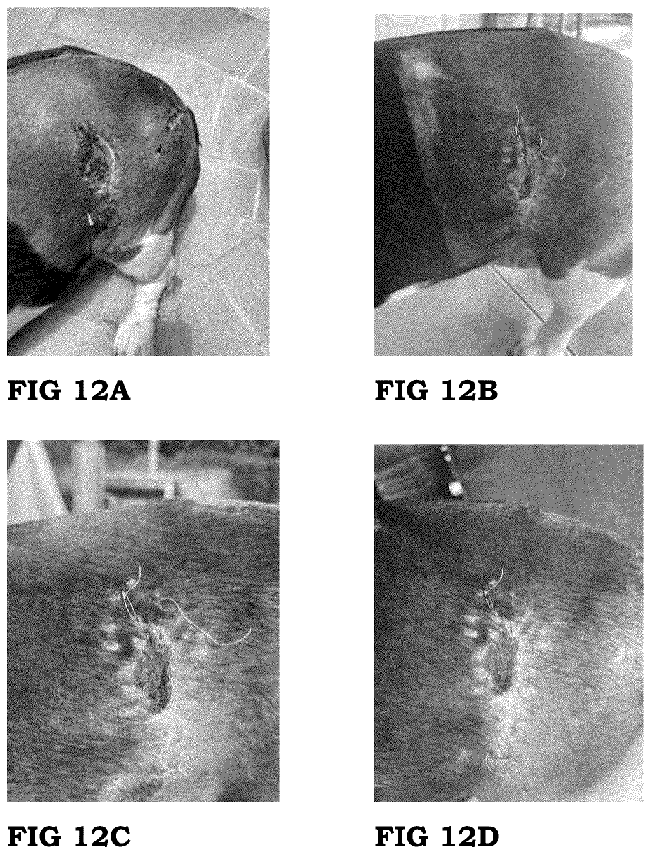

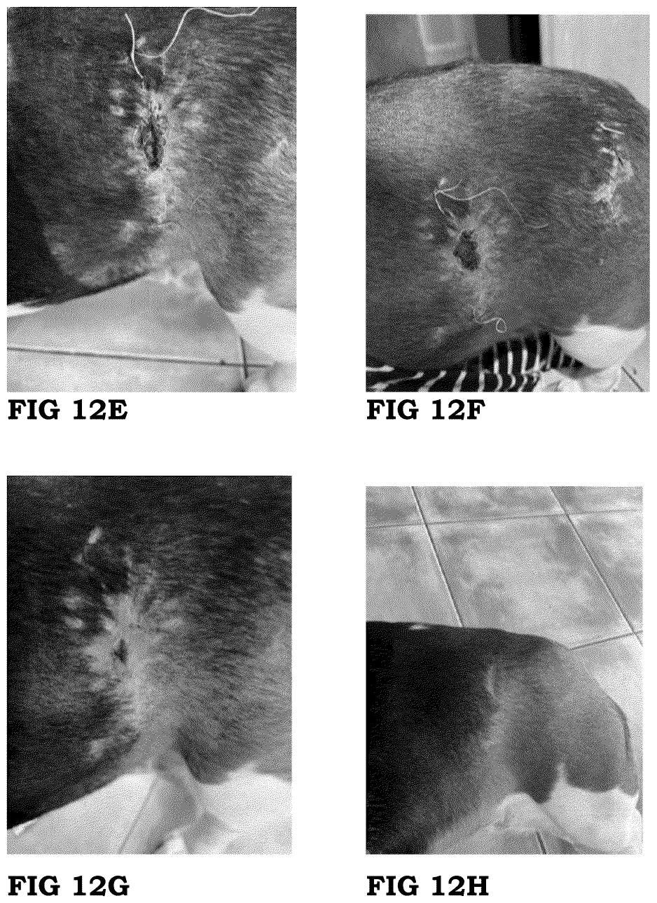

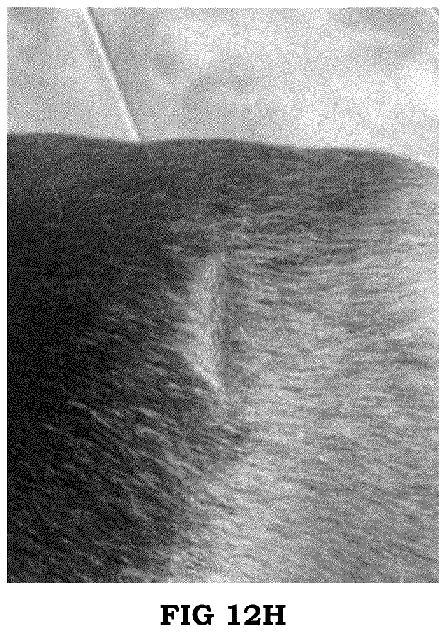

FIGS. 12A-12H illustrate an example of a wound care application to a dog. In this example, the dog is a basset hound. Two to three pumps of the treatment composition to the wound site twice daily. The wound site was left uncovered and open to the environment. FIG. 12A illustrates the wound site at the time of the injury, which will be defined as Day 1. The healing process on Day 4 is illustrated in FIG. 12B. The healing process on Day 5 is illustrated in FIG. 12C. The healing process at Day 6 is illustrated in FIG. 12D. The healing process on Day 7 is illustrated in FIG. 12E. The healing process on Day 8 is illustrated in FIG. 12F. The healing process at Day 9 is illustrated in FIG. 12G. The wound is completely healed on Day 10, as illustrated in FIGS. 121 and 12H.

### Example 3

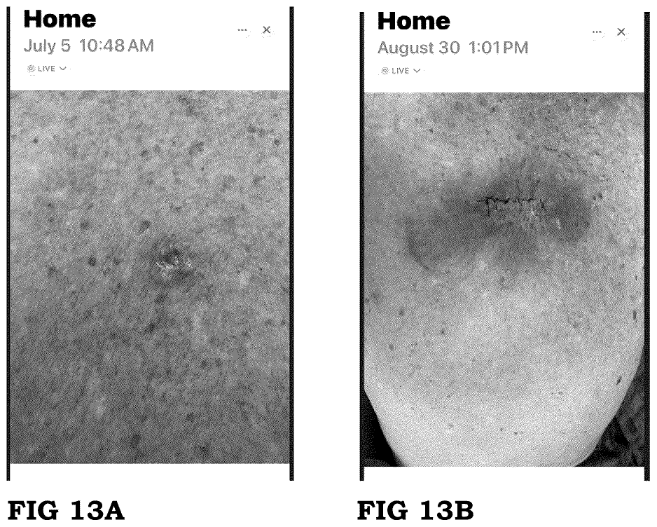

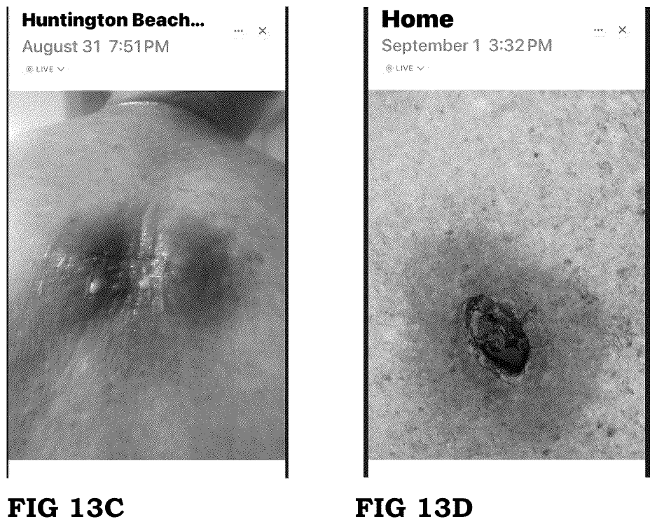

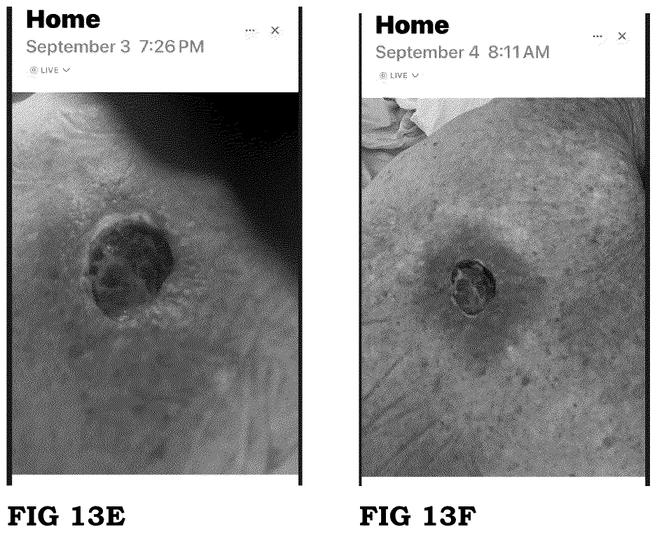

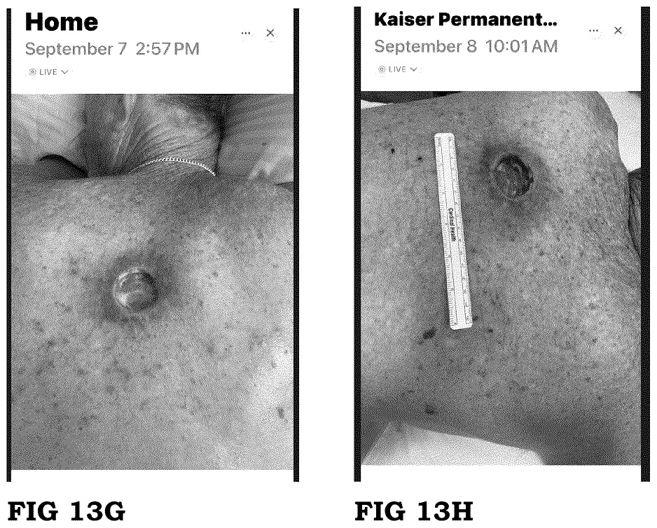

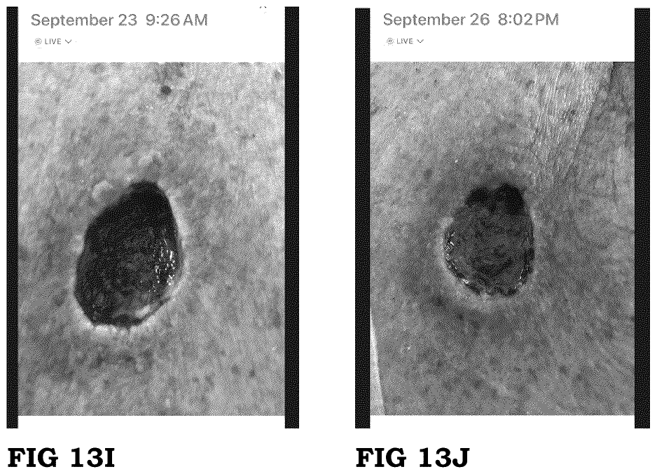

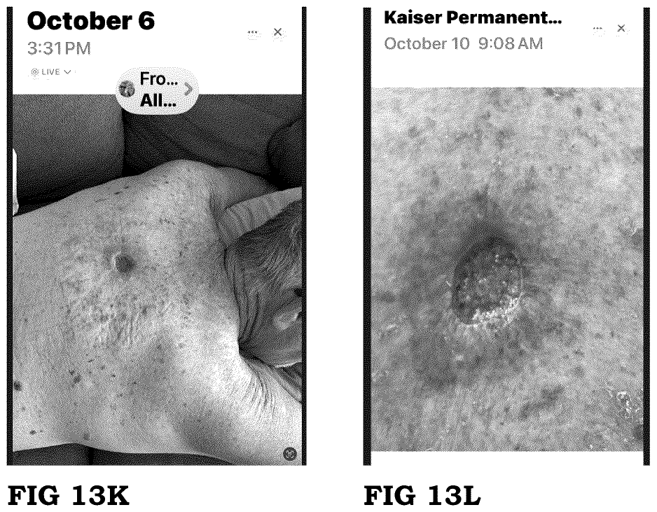

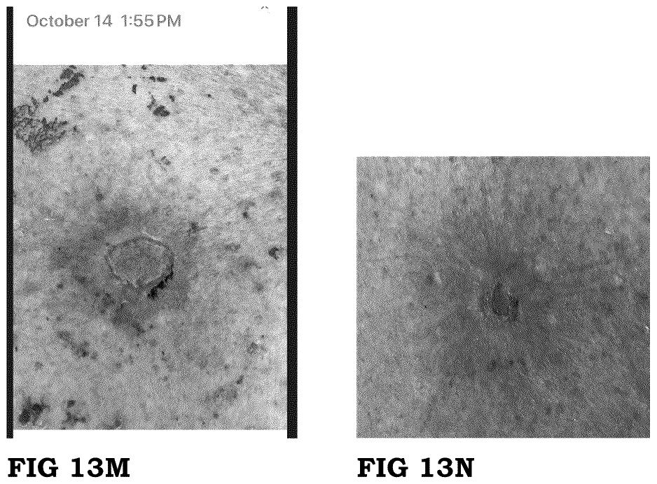

With reference to FIGS. 13A-13N, an example of wound care is illustrated. The subject was diagnosed with basal cell carcinoma in July and underwent excisional surgery on August 25th. Three days post-operatively, an infection developed at the surgical site. To manage this, the wound was reopened, thoroughly cleaned, and left open to heal by secondary intention. The subject was instructed to adhere to strict wound care, including cleaning and packing the wound three times daily with Aquacel advanced antimicrobial dressing. Beginning on September 16th, a sprayable bandage system, in accordance with the present invention, was applied daily before wound packing to support the healing process.

FIG. 13A illustrates the treatment area pre-surgery. FIG. 13B illustrates the treatment area post-surgery. FIG. 13C illustrates the infection in the treatment area. FIG. 13D illustrates the treatment area after the wound was reopened. FIGS. 13E-13H illustrate the slow healing process before September 16th, which is the date that the treatment using the sprayable bandage system commenced. FIGS. 13I-13N illustrate the acerated healing process from the treatment using the sprayable bandage system, in accordance with the present invention,

Various embodiments provide a wound dressing device, comprising a biocompatible matrix comprising hyaluronic acid and at least one silane, wherein the at least one silane is cross-linked to the hyaluronic acid; wherein the biocompatible matrix is insoluble; and wherein the biocompatible matrix is an antimicrobial.

In some embodiments, the wound dressing device can further comprise a woven material. In some examples, the biocompatible matrix is coated onto the woven material. In some examples, the woven material is a gauze. In some examples, the woven material further comprises a bandage.

In some embodiments, the wound dressing device can further comprise an active agent directly incorporated into the biocompatible matrix. In some examples, the active agent is an alcohol. In some examples, the active agent is a medicant.

In some embodiments, the biocompatible matrix is a film or a sheet. In some embodiments, the wound dressing device can comprise an adhesive configured for an attachment of the biocompatible matrix to a skin surface.

In some embodiments, the at least one silane is 1-Octadecanaminium, N,N-dimethyl-N-[3-(trihydroxysilyl) propyl]. In some embodiments, the at least one silane comprises a quaternary ammonium group.

In some embodiments, the wound dressing device can further comprise a non-woven material. In some examples, the biocompatible matrix is coated onto the non-woven material. In some examples, the non-woven material is a sponge. In some embodiments, wound dressing device comprises at least one of a foam dressing, an Alginate dressing, and a transparent dressing.

Various embodiments provide methods for treating wounds. In some embodiments, a method comprises administering a wound healing device comprising a biocompatible matrix comprising matrix comprising hyaluronic acid and at least one silane, wherein the at least one silane is cross-linked to the hyaluronic acid; and wherein the biocompatible matrix is an antimicrobial.

In some examples, the at least one silane is 1-Octadecanaminium, N,N-dimethyl-N-[3-(trihydroxysilyl) propyl]. In some examples, the at least one silane comprises a quaternary ammonium group.

In some embodiments, the method can further comprise an active agent directly incorporated into the biocompatible matrix. In some examples, the active agent is an alcohol. In some examples, the active agent is selected from the group consisting of antimicrobial agents, antifungal agents, antiviral agents, metals and wound healing agents.

In some examples, the wound healing agents are selected from the group consisting of growth factors, mucopolysaccharides and proteins. In some examples, the growth factor is selected from the group consisting of fibroblast growth factor (bFGF), acidic fibroblast growth factor (aFGF), nerve growth factor (NGF), epidermal growth factor (EGF), insulin-like growth factors 1 and 2, (IGF-1 and IGF-2), platelet derived growth factor (PDGF), tumor angiogenesis factor (TAF), vascular endothelial growth factor (VEGF), corticotropin releasing factor (CRF), transforming growth factors α and β (TGF-α and TGF-β), interleukin-8 (IL-8); granulocyte-macrophage colony stimulating factor (GM-CSF), interleukins, and interferons.

In some embodiments, the method can further comprise applying a woven material to the wounds, wherein the biocompatible matrix further comprises the woven material. In some examples, the biocompatible matrix is coated onto the woven material.

In some embodiments, the method can further comprise adhering the biocompatible matrix to skin surrounding the wounds, wherein the biocompatible matrix further comprises an adhesive.

In some embodiments, the method can further comprise applying a non-woven material to the wounds, wherein the biocompatible matrix further comprises the non-woven material.

In some embodiments, the method can further comprise adhering the biocompatible matrix to skin surrounding the wounds, wherein the biocompatible matrix further comprises an adhesive.

In some embodiments, the method can further comprise absorbing at least one of water and exudate from the wounds into the biocompatible matrix.

In some embodiments, the method can further comprise disrupting growth of microorganisms in the wounds.

As used herein, the phrase “at least one of A, B, and C” can be construed to mean a logical (A or B or C), using a non-exclusive logical “or;” however, it can be construed to mean (A, B, and C); in addition, it can be construed to mean (A and B) or (A and C) or (B and C). As used herein, the phrase “A, B and/or C” should be construed to mean (A, B, and C) or alternatively (A or B or C), using a non-exclusive logical “or.”

The present invention has been described above with reference to various exemplary embodiments and examples, which are not intended to be limiting in describing the full scope of systems and methods of this invention. However, those skilled in the art will recognize that equivalent changes, modifications and variations of the embodiments, materials, systems, and methods may be made within the scope of the present invention with substantially similar results and are intended to be included within the scope of the present invention, as set forth in the following claims.

1. A wound dressing device, comprising
a biocompatible matrix comprising hyaluronic acid and at least one silane,
wherein the at least one silane is cross-linked to the hyaluronic acid;
wherein the biocompatible matrix is insoluble; and
wherein the biocompatible matrix is an antimicrobial.

2. The wound dressing device according to claim 1, further comprises a woven material.

3. The wound dressing device according to claim 2, wherein the biocompatible matrix is coated onto the woven material.

4. The wound dressing device according to claim 2, wherein the woven material is a gauze.

5. The wound dressing device according to claim 2, wherein the woven material further comprises a bandage.

6. The wound dressing device according to claim 1, further comprises an active agent directly incorporated into the biocompatible matrix.

7. The wound dressing device according to claim 6, wherein the active agent is an alcohol.

8. The wound dressing device according to claim 6, wherein the active agent is a medicant.

9. The wound dressing device according to claim 1, wherein the biocompatible matrix is a film.

10. The wound dressing device according to claim 1, further comprises an adhesive configured for an attachment of the biocompatible matrix to a skin surface.

11. The wound dressing device according to claim 1, wherein the at least one silane is 1-Octadecanaminium, N,N-dimethyl-N-[3-(trihydroxysilyl) propyl].

12. The wound dressing device according to claim 1, wherein the at least one silane comprises a quaternary ammonium group.

13. The wound dressing device according to claim 1, further comprises a non-woven material.

14. The wound dressing device according to claim 13, wherein the biocompatible matrix is coated onto the non-woven material.

15. The wound dressing device according to claim 13, wherein the non-woven material is a sponge.

16. A method for treating wounds, comprising administering a wound healing device comprising a biocompatible matrix comprising matrix comprising hyaluronic acid and at least one silane, wherein the at least one silane is cross-linked to the hyaluronic acid; and wherein the biocompatible matrix is an antimicrobial.

17. The method according to claim 16, wherein the at least one silane comprises a quaternary ammonium group.

18. The method according to claim 16, further comprises an active agent directly incorporated into the biocompatible matrix.

19. The method according to claim 17, wherein the active agent is an alcohol.

20. The method according to claim 17, wherein the wherein the active agent is selected from the group consisting of antimicrobial agents, antifungal agents, antiviral agents, metals and wound healing agents.

21. The method according to claim 20, wherein the wound healing agents are selected from the group consisting of growth factors, mucopolysaccharides and proteins.

22. The method according to claim 21, wherein the growth factor is selected from the group consisting of fibroblast growth factor (bFGF), acidic fibroblast growth factor (aFGF), nerve growth factor (NGF), epidermal growth factor (EGF), insulin-like growth factors 1 and 2, (IGF-1 and IGF-2), platelet derived growth factor (PDGF), tumor angiogenesis factor (TAF), vascular endothelial growth factor (VEGF), corticotropin releasing factor (CRF), transforming growth factors α and β (TGF-α and TGF-β), interleukin-8 (IL-8); granulocyte-macrophage colony stimulating factor (GM-CSF); the interleukins, and the interferons.

23. The method according to claim 16 further comprises applying a woven material to the wounds, wherein the biocompatible matrix further comprises the woven material.

24. The method according to claim 23, wherein the biocompatible matrix is coated onto the woven material.

25. The method according to claim 23 further comprises adhering the biocompatible matrix to skin surrounding the wounds, wherein the biocompatible matrix further comprises an adhesive.

26. The method according to claim 16, further comprises applying a non-woven material to the wounds, wherein the biocompatible matrix further comprises the non-woven material.

27. The method according to claim 26 further comprises adhering the biocompatible matrix to skin surrounding the wounds, wherein the biocompatible matrix further comprises an adhesive.

28. The method according to claim 16 further comprises absorbing water from the wounds into the biocompatible matrix.

29. The method according to claim 16 further comprises disrupting growth of microorganisms in the wounds.

30. The method according to claim 16, wherein the at least one silane is 1-Octadecanaminium, N,N-dimethyl-N-[3-(trihydroxysilyl) propyl].
